SEQUENCE LISTING (3 sequences)

SEQ ID NO: 1 - protein, 11 aa, synthetic construct
TVVTLIGVVTI

SEQ ID NO: 2 - DNA, 45 bp, synthetic construct
  source 1..45: mol_type = other DNA
acagttgtcacgctgattggtgtcgttacaatctaacgcatcgcc

SEQ ID NO: 3 - DNA, 45 bp, synthetic construct
  source 1..45: mol_type = other DNA
ggcgatgcgttagattgtaacgacaccaatcagcgtgacaactgt

(19) United States
(12) Patent Application Publication
(10) Pub. No.: US 2026/0301875 A1
(43) Pub. Date: Oct. 1, 2026

(54) METHODS TO IDENTIFY NOVEL GENETIC FEATURES ASSOCIATED WITH A TARGETED PHENOTYPE BY DISCOVERY-ORIENTED MACHINE LEARNING ANALYSIS OF A GENOME ASSEMBLY OR DATASET

(71) Applicant: The Regents of the University of California, Oakland, CA (US)

(72) Inventors: Jason C. Hyun, Rockville, MD (US); Bernhard Orn Palsson, San Diego, CA (US)

(21) Appl. No.: 19/477,702

(22) PCT Filed: May 7, 2024
(86) PCT No.: PCT/US2024/028202

§ 371 Date: Oct. 22, 2025

Related U.S. Application Data
(60) Provisional application No. 63/464,926, filed on May 8, 2023.

(51) Int. Cl. G16B 40/20; G06N 20/00; G16B 20/20; G16B 30/00

(52) U.S. Cl. CPC G16B 40/20; G06N 20/00; G16B 20/20; G16B 30/00

(57) ABSTRACT

The disclosure provides methods to identify novel genetic features associated with a targeted phenotype (e.g., antimicrobial resistance) by discovery-oriented machine learning analysis of a dataset of genome assemblies and corresponding measurements related to the phenotype.

### CROSS REFERENCE TO RELATED APPLICATIONS

This application claims priority under 35 U.S.C. § 119 from Provisional Application Ser. No. 63/464,926, filed May 8, 2023 the disclosure of which is incorporated herein by reference.

### STATEMENT OF GOVERNMENT SUPPORT

This invention was made with Government support under AI124316, awarded by the National Institutes of Health. The Government has certain rights in the invention.

### TECHNICAL FIELD

The disclosure provides methods to identify novel genetic features associated with a targeted phenotype (e.g., antimicrobial resistance) by discovery-oriented machine learning analysis of a dataset of genome assemblies and corresponding measurements related to the phenotype.

### INCORPORATION BY REFERENCE OF SEQUENCE LISTING

Accompanying this filing is a Sequence Listing entitled, “00015-426WO1.xml” created on May 7, 2024 and having 3,944 bytes of data, machine formatted on IBM-PC, MS-Windows operating system. The sequence listing is hereby incorporated by reference in its entirety for all purposes.

### BACKGROUND

Antimicrobial resistance (AMR) remains a persistent problem in the treatment of bacterial infections. With resistance having been observed against nearly all major antibiotics, 700,000 annual deaths are currently attributable to AMR globally and is projected to increase to as high as 10 million by 2050 without major interventions. One strategy for managing AMR is the large-scale sequencing of infection isolates, which has yielded tens of thousands of publicly available genome sequences for each major bacterial pathogen and frequently paired with resistance metadata.

This wealth of data has enabled global analyses on the genetics of AMR, many of which employ machine learning (ML) to predict AMR phenotypes directly from genetic variations. Accurate AMR phenotype prediction models trained on thousands of genomes have been developed for many pathogens such as Escherichia coli, Klebsiella pneumoniae, Mycobacterium tuberculosis, Salmonella enterica, or multiple species, with numerous others developed from smaller datasets. However, many of these studies report a significant fraction of their models' predictive genetic features to have no relationship to known AMR mechanisms. This disconnect between statistically identified and mechanistically established genetic determinants of AMR highlights the current gap in knowledge in AMR genetics and remains a challenge for the real world adoption of ML-based systems for rapidly predicting AMR and informing treatment strategies.

### SUMMARY

Surveillance programs for managing antimicrobial resistance (AJMR) have yielded thousands of genomes suited for data-driven mechanism discovery. A workflow is presented herein which integrates pangenomics, gene annotation, and machine learning to identify known and novel AMR genes at scale. Applied to 12 species spanning 27,155 genomes and 69 drugs, it was demonstrated herein that discovery-oriented support vector machines outperform conventional methods at recovering known AMR genes, by recovering 263 genes compared 145 by Pyseer and 125 by Fisher's exact test, and further identifying 142 novel AMR gene candidates. Validation of two candidates revealed cases of conditional resistance: ΔcycA conferred ciprofloxacin resistance in minimal media with D-serine, and frdD V111D conferred ampicillin resistance in the presence of ampC by modifying the overlapping promoter. It is expected that this approach is adaptable to other species and phenotypes.

In a particular embodiment, the disclosure provides a computer-implemented method to identify novel genetic features that are predictive of a phenotype based upon a genome collection indicative of a genus, family or species of organism of interest from a dataset of genome assemblies and corresponding measurements related to the phenotype, comprising carrying out steps (1-4) and optionally, step 1′: (1) enumerating genetic variation through pangenome construction from genome assemblies; (1′) identifying genetic features known to be associated with a phenotype to supplement the machine learning models of steps (2)-(3); (2) training machine learning models to predict phenotypes from the genetic variation enumerated in step (1) and evaluating the machine learning model for best performing hyperparameter (HP) ranges predictive of a phenotype from a genetic feature (s), based on phenotype prediction accuracy and/or recovery of genetic features known to be associated with the phenotype; (3) re-training the machine learning model of step (2) with best performing hyperparameter (HP) ranges and sorting genetic features that are most predictive of a phenotype based upon a genome collection indicative of a genus, family or species of organism of interest in order to generate a defined, ranked set of genetic features predictive of a phenotype based upon a genome collection indicative of a genus, family or species of organism of interest; and (4) identifying novel genetic features that are predictive of a phenotype based upon a genome collection indicative of a genus, family or species of organism of interest by removing features already known to be predictive of a phenotype based upon a genome collection indicative of a genus, family or species of organism of interest from the defined, ranked set of genetic features in step (3). In a further embodiment, the phenotype of interest is antimicrobial resistance. In yet a further embodiment, the dataset comprises genome assemblies from microbes. In another embodiment, for step (1), the genome assemblies are first processed by carrying out steps (a)-(d), and optionally, step (d′): (a) identifying genetic features in each genome assembly; (b) dividing identified genetic features into protein coding sequences (CDSs) and noncoding features; (c) computing the number of CDSs, number of contigs, and total length of each genome assembly in the dataset; (d) identifying genomes assemblies that meet defined genetic feature criteria by filtering out genome assemblies from the dataset that do not satisfy one or more of the following criteria: (i) the number of contigs is within 2.5 times the median across all genome assemblies, (ii) the number of CDs is within 3 standard deviations of the mean across all genome assemblies, and (iii) the total genome length is within 3 standard deviations of the mean across all genome assemblies; and (d′) filtering the identified genome assemblies of (d) to filter out genomes that have an EvalCon fine consistency less than 90%, less than 88%, less than 87%, less than 85% or less than 80%. In yet another embodiment, genome assemblies are filtered out from the dataset if they do not meet criteria (i), (ii) and (iii). In a certain embodiment, for step (1), the pangenomes are constructed and genetic variation is enumerated thereof, by carrying out steps (A)-(F) and optionally step (D′): (A) reducing the set of all identified CDSs across all genome assemblies to a non-redundant set of unique CDSs; (B) clustering all unique CDSs by amino acid sequence; (C) enumerating every CDS cluster, and every sequence variant of each CDS cluster, wherein the enumerated CDS clusters are referred to as “genes”, and wherein sequence variants of each enumerated CDS cluster are referred to as “gene coding variants” of the corresponding gene; (D) identifying unique noncoding sequences flanking occurrences of each gene; (D′) if noncoding nucleic acid sequence features are available, then steps (A)-(C) are repeated for noncoding nucleic acid sequences to yield enumerated noncoding sequence clusters and variants, wherein the enumerated noncoding clusters are referred to as “noncoding features”, and wherein sequence variants of each enumerated noncoding cluster are referred to as “noncoding variants;” (E) determining the presence/absence of features (1)-(4) and optionally (5) and (6) for all genome assemblies: (1) genes, (2) gene coding variants, (3) gene 5′ variants, (4) gene 3′ variants, (5) noncoding features, and (6) noncoding variants; and (F) encoding the genetic variation of the dataset as a (genome assembly x feature) binary matrix of these presence/absence calls. In another embodiment, for step (D), the unique noncoding sequences flanking the occurrences of the gene are identified by: (i) identifying the locations of gene coding variants across genome assemblies; (ii) extracting 300 bp sequences immediately upstream and/or downstream at each location; (iii) removing sequences shorter than 300 bp due to contig boundaries; and (iv) reducing all upstream and/or downstream sequences into non-redundant sets of 300 bp nucleic acid sequences, wherein the non-redundant sets of 300 bp nucleic acid sequences are referred to as gene 5′ variants and gene 3′ variants, respectively. In yet another embodiment, the computer-implemented method includes step (1′), and wherein the genetic features known to be associated with a phenotype to supplement the machine learning models of step (2) are identified by carrying out steps (I)-(IV): (I) Identifying the most common coding variant for each gene, and annotating the variants for phenotype-associated genes; (II) Extracting the phenotype-associated genes based upon defined criteria; (III) Identifying other genes that have same defined criteria as the phenotype-associated genes, and annotate these other genes as phenotype-associated genes; and (IV) Labelling all phenotype-associated genes, as well as their associated coding variants, 5′ variants, and 3′ variants as genetic features that are known to be associated with a phenotype and species of interest. In a further embodiment, the phenotype-associated genes contribute to antimicrobial resistance. In yet a further embodiment, the variants are annotated as antimicrobial resistance (AJAR) genes using RGI from CARD. In another embodiment, the genes annotated as AMR genes are assigned an ARO ID, and are extracted based upon the criteria of being associated with a drug of interest based upon the CARD ontology, and wherein the extraction process comprises: (aa) initializing a graph with each ARO ID from the CARD ontology corresponding to a node, and adding an edges U→V whenever: (i) U is a gene and has relationship “is_a” to V; (ii) V is a drug and has relationship “is_a” to U; (iii) V has relationship “has_part” to U; (iv) U has any of the following relationships to V: “part_of”, “regulates”, “confers_resistance_to_antibiotic”, “confers_resistance_to_drug_class”; and (bb) filtering the initial identified AMR genes to whose ARO ID has a direct path to the node corresponding to the drug of interest in the graph. In another embodiment, for step (2), the machine learning model comprises the following methodology: identifying the top 50,000 genetic features by individual association with the phenotype of interest, sorting by log odds ratio (LOR), and acquiring the top 25,000 and bottom 25,000 genetic features; defining the machine learning model and hyperparameter (HP) ranges to use in predicting the phenotype, training and evaluating the model and HP combinations over x-fold cross validation, wherein the accuracy for an HP combination is defined as the mean Matthews correlation coefficient (MCC) on the test set across the x-folds; and selecting the HP combination that yields the best performance over the x-fold cross validation, wherein the HP combination with the highest mean MCC is selected. In a further embodiment, the machine learning model uses 5-fold cross validation. In yet a further embodiment, the machine learning model is support vector machine (SVM) ensembles implemented in scikit-learn. In another embodiment, the HP ranges are defined as follows: (aaa) number of estimators=25, 50, 100, 200; (ii) fraction of samples per estimator=25%, 50%, 75%, 100%; (bbb) fraction of features per estimator=25%, 50%, 75%, 100%; and (ccc) C (SVM regularization term)=0.1, 1, 10, 100. In a further embodiment, if the computer-implemented model includes step (1′), then evaluating how the known genetic features were recovered by the machine learning model at each fold using the following: (xx) for a trained model at each fold, computing each genetic feature's weight as the absolute value of the average of coefficients assigned to a genetic feature across all individual SVMs in the ensemble that had access to the feature, when accounting from genetic feature subsampling; (yy) sorting and ranking all features by weight, and computing a model “GWAS Score” from the ranks “r” of known phenotype-associated features using EQ. 1:

$$GWASScore = {\sum }_{r ∈ known} 0 . {5}^{( r - 1 ) / 10} \qquad ( Eq . 1 )$$

wherein rank=1 corresponds to the highest weight; and (zz) computing the mean GWAS Score across the x folds as the GWAS Score of the HP combinations. In yet another embodiment, if known features were annotated, then each HP combination is ranked by mean MCC and separately by mean GWAS Score and selecting the HP combination with the highest sum of ranks, wherein rank=1 corresponds to the highest MCC or GWAS Score. In a further embodiment, the machine learning model predicts a binary phenotype variable. In an alternate embodiment, the machine learning model is modified to predict categorical or continuous/numerical variables.

### DESCRIPTION OF DRAWINGS

FIG. 1A-E provides genomic and antimicrobial resistance datasets assembled from the PATRIC database for 12 pathogenic species. (A) Workflow for extracting six types of biological features from a species' genome collection. For each species, the number of (B) high-quality genomes with AMR data, (C) experimentally measured susceptible-intermediate-resistant (SIR) data points (directly reported or inferred from reported minimum inhibitory concentrations; MICs) with consistent testing standards across all drugs, and (D) number of drugs for which SIR data is available is shown. Species have been sorted by number of genomes. The macrolide-lincosamide-streptogramin B drug class is abbreviated MLSB. (E) Number of known AMR genes identified for each species-drug case. Drugs are sorted by drug class, and species are sorted by total number of AMR gene-drug mappings identified.

FIG. 2A-C provides cross-species analysis of 68,324 known antimicrobial resistance gene alleles identified during the annotation stage of the workflow and gene locations. (A) Relationship between the number of species an AMR allele is observed in and tendency to be plasmid-encoded. (B) Number of AMR alleles shared between each pair of species, compared to species phylogenetic class. (C) Distribution of predicted gene locations and total occurrences per phylogenetic class for AMR alleles appearing in at least 10 genomes in multiple classes.

FIG. 3A-D presents cross-species analysis of 6,332 antimicrobial resistance genes, gene locations, and functions. (A) Relationship between the number of species an AMR gene is observed in and tendency to be plasmid-encoded. (B) Enrichment of AMR gene functional categories in plasmid—over chromosomally-encoded genes and in multispecies over single species genes, based on log 2 odds ratios (LORs). (C) Number of AMR genes shared between each pair of species, compared to species phylogenetic class. (D) Distribution of predicted gene locations and total occurrences per phylogenetic class for AMR genes appearing in at least 10 genomes of multiple classes. The three blaTEM cluster columns correspond to different sequence clusters identified by CD-HIT all related to TEM family beta-lactamases, with the first representing full variants and the others representing fragments.

FIG. 4A-E presents an evaluation of a GWAS-oriented machine learning workflow for identifying AMR-associated genetic features in 127 species-drug cases. (A) Workflow integrating support vector machine (SVM) ensembles, hyperparameter optimization, and AMR gene annotation to train models suited for both phenotype prediction and AMR gene identification from genome assemblies and SIR phenotype data. (B) Performance of models for 127 species-drug cases, by mean test set Matthews correlation coefficient (MCC) during 5-fold cross validation and recovery of known AMR genes. (C) Comparison between SVMs and Pyseer at recovering known AMR genes. (D) Total known AMR gene-drug mappings recovered by SVMs, Pyseer, and Fisher's exact tests across all cases. (E) Rankings of known AMR genes recovered among SVMs' top features, grouped by whether or not the gene was also recovered by Pyseer or Fisher's exact test.

FIG. 5 demonstrates the Impact of SVM ensemble hyperparameters on AMR phenotype prediction performance and recovery of known AMR genes across 256 hyperparameter combinations and 10 species-drug cases. Each row corresponds to a single AMR prediction problem between a species and a drug, and each column corresponds to a varied hyperparameter. X-axes show average MCCs on the test set from 5-fold cross validation (CV) experiments, y-axes show average GWAS scores across the CV experiment.

FIG. 6A-B presents the overall impact of SVM ensemble hyperparameters on performance and global performance of hyperparameter-optimized SVM ensembles. (A) Kruskal-Wallis ANOVA tests between hyperparameters and either AMR phenotype prediction performance (Matthew's correlation coefficient; MCC) or biological relevance (GWAS score) across 10 representative species-drug cases. (B) Impact of feature subsampling on model performance for representative cases. MCCs and GWAS scores have been normalized to mean 0 and standard deviation 1, within their specific species-drug cases.

FIG. 7 displays performance improvements from hyperparameter optimization of SVM ensembles across 127 species-drug cases. Performance by three metrics is shown for SVM ensembles trained using default fixed hyperparameters (C=1, feature fraction=50%, sample fraction=75%, ensemble size=50) compared to ensembles with hyperparameters optimized to balance both phenotype prediction accuracy and known AMR gene recovery. Performance is based on means from 5-fold cross validation

FIG. 8A-B provides identification of novel AMR gene candidates from machine learning models. (A) Candidate identification workflow. From each trained AMR-ML model, the top 10 predictive features were identified, filtered for features that are neither known AMR genes nor very rare, ranked based on statistical evidence for resistance in other related drugs, and finally categorized by functional annotation. When multiple features related to the same gene were predictive of resistance, the feature with the strongest evidence was selected and the others were labeled as correlates. Mobile genetic element is abbreviated MGE. (B) Distribution of 43 well-characterized AMR gene candidates by species and drug class.

FIG. 9A-D shows D-serine-dependent impact of amino acid transporter CycA on quinolone resistance. (A) Enrichment for resistant over susceptible genomes across observed cycA variants for four quinolone drugs, based on log₂ odds ratio (LOR). Number of genomes with AMR data is shown for each drug, and only variants observed in at least 100 genomes are shown. (B) Maximum cell density (OD600) achieved by E. coli BW25113 wildtype (WT) vs. cycA knockout (KO) across 60 conditions from combinations between media, ciprofloxacin (CIP) concentration, and CycA substrate supplementation. Error bars indicate standard deviations centered around means from biologically independent triplicates. Conditions where density differs significantly between WT and KO are labeled (n_{WT}=n_{KO}=3, two-sided Welch t-test, Benjamini-Hochberg correction, FDR <0.05). (C) Absolute (above) and relative (below) maximum densities between WT and KO in conditions involving D-serine. Starred conditions correspond to the same significant differences from FIG. 9B. Error bars indicate standard deviations centered around means from biologically independent triplicates as in FIG. 9B for absolute densities. Relative densities show ratios between means from absolute densities. Significant differences between WT and KO are starred (p=0.00040, 0.00344, and 0.00004 for CIP concentrations in M9 of 16, 62, and 125 μg/L, respectively). (D) Possible model for interactions between D-serine, cycA, media, and fluoroquinolones consistent with the observed conditional resistance conferred by cycA KO.

FIG. 10A-C demonstrates ampC-dependent beta-lactam resistance conferred by the V111D substitution in fumarate reductase subunit frdD. (A) Enrichment for resistant over susceptible genomes from the presence of frdD mutations resulting in the V111D substitution across 14 beta-lactam drugs, based on log₂ odds ratio (LOR). Number of genomes with AMR data is shown for each drug. (B) Overlap between the frdD coding region and ampC promoter in E. coli BW25113 and predicted ampC transcription rates for various frdD V111 mutations (SEQ ID NOs: 1-3 in order of appearance). (C) Maximum cell density (OD600) achieved by ampC and frdD mutants under increasing ampicillin (AMP) concentrations in rich (CA-MHB) or minimal (M9) media. Error bars indicate standard deviations centered around means from biologically independent triplicates. Six genotypes were tested, from combinations between three possible codons at frdD V111 in either the BW25113 wildtype (WT) or corresponding ampC knockout (KO) strain. Starred case indicates growth was not observed until at least 8 hours after inoculation for all replicates.

FIG. 11A-B shows diversity of samples by subtype, study of origin, and AMR phenotype. (A) Distribution of MLST subtypes and BioProject accession IDs for each species' genome collection. For MLSTs, the total number of additional MLST subtypes beyond the five most common are labeled. BioProjects comprising at least 50% of genomes for a given species are labeled. (B) Distribution of AMR phenotypes by species, drug, and drug class. Drugs are sorted by drug class. Macrolide-lincosamide-streptogramin B is abbreviated “MLSB”.

FIG. 12A-D shows distribution of TEM-family beta-lactamases observed in 4,861 genomes across 8 species. (A) Distribution of TEM-family beta-lactamase (blaTEM) alleles with respect to genome count, harboring species, and mutations relative to the TEM-1 allele. Alleles occurring in at least 10 genomes are labeled. TEM* and TEM** refer to unnamed blaTEM alleles. (B) Alignments between contigs from genomes with TEM-116 and PLSDB plasmids based on MASH distance. Known plasmids carrying blaTEM are indicated. All plasmids with MASH distance <0.025 to at least one genome with TEM-116 are shown. (C) Distribution of the three plasmids containing TEM-116 by species. (D) Relationship between beta-lactam associated AMR genes and cefoxitin minimum inhibitory concentration (MIC) in S. aureus. Numeric labels correspond to the number of genomes observed with the corresponding AMR genes and MIC.

FIG. 13A-D shows sequence similarity between S. enterica and S. aureus genomes carrying TEM-116. (A-B) Distributions of pairwise MASH distances between all 3,302 S. enterica and 2,248 S. aureus genomes. (C-D) Clustermaps based on pairwise MASH distances between the 17 S. enterica and 11 S. aureus genomes carrying TEM-116. Heatmaps share the same color scales. Genomes are colored by their predicted blaTEM plasmid and are clustered using single linkage and Euclidean distances.

FIG. 14 shows cefoxitin minimum inhibitory concentration (MIC) versus beta-lactam resistance-associated genes across 38 S. aureus genomes. Rows and columns have been ordered by hierarchical clustering with Jaccard distances and average linkage.

FIG. 15A-C shows generalizability of the GWAS score. For each species-drug case, half of all known AMR genes were hidden randomly and GWAS scores were computed using either visible or hidden AMR genes for models trained using each possible hyperparameter combination. The distribution of Spearman correlations between the two GWAS scores is shown in (A) for n=100 random selections of hidden AMR genes. Boxplots indicate Q1, median, Q3, whiskers at maximum and minimum values within 1.5*IQR of the median, and outliers outside this range. (B) Standard deviation of GWAS scores per species-drug case. (C) Distribution of GWAS score Spearman correlations by species-drug case.

FIG. 16A-B shows relationship between dataset parameters and model performance across 127 species-drug cases. (A) Performance of hyperparameter-optimized SVM ensembles vs. dataset size, extent of class imbalance, abundance of “intermediate” resistant genomes, and total known AMR genes annotated. Spearman correlation coefficients across the n=127 species-drug cases are shown. (B) Performance of the 127 SVM ensembles versus drug class.

FIG. 17A-C shows visualization of the top 20 genetic features associated with AMR for three species-drug cases as identified by SVM ensembles. Top features are shown for cases (A) A. baumannii vs. amikacin, (B) E. coli vs. ciprofloxacin, and (C) K. pneumoniae vs. imipenem. Features related to known AMR genes are colored and also labeled by whether they were also recovered by Pyseer and/or Fisher's exact test, and other features are black. Features related to undercharacterized genes are labeled as either “hypothetical protein” or “mobile element”, numbered by the order the gene is displayed. The type of genetic variant is also shown for each feature.

FIG. 18A-C shows additional comparisons between performance of SVM ensembles, Pyseer, and Fisher's exact test at recovering known AMR genes. (A) Comparison between SVMs and Fisher's exact test at recovering known AMR genes across 127 species-drug cases. (B-C) Total known AMR gene-drug mappings recovered by SVMs, Pyseer, and Fisher's exact tests across all cases, when defining a recovered gene as those in the top 10 or top 50 features when sorting by feature weight (for SVM) or p-value (for Pyseer and Fisher's exact test).

FIG. 19A-D shows associations between known AMR features and resistance. For each known AMR feature in a given species-drug case, the fraction of genomes with the feature that are resistant to the drug was computed. The distribution of these resistance fractions combined across 127 species-drug cases is shown in (A), with black dots representing rare features (found in no more than 2 genomes) and blue bars representing all other known AMR features. The tendency for these distributions to concentrate near 0 and 1 was quantified by computing means for values (B) less than or (C) greater than 0.5. Individual distributions for 10 species-drug cases are shown in (D).

FIG. 20 shows the impact of varying the preliminary feature filter on downstream model performance. Each row shows the effect of the feature filter on a model performance metric, either the maximum or median test set MCC (from 5-fold CV) or GWAS score across 256 hyperparameter combinations. The first column shows model performance when limited to the top 5,000, 10,000, 20,000, or 50,000 features after sorting by log odds ratio (LOR) for resistance, and the second column shows model performance under analogous filters sorting instead by Fisher's exact test p-value for resistance. The third column compares performance between LOR and Fisher's exact test filters for identical feature count limits. Each color represents results for a specific species-drug case.

### DETAILED DESCRIPTION

As used herein and in the appended claims, the singular forms “a,” “an,” and “the” include plural referents unless the context clearly dictates otherwise. Thus, for example, reference to “a gene” includes a plurality of such genes and reference to “the gene variant” includes reference to one or more gene variants and equivalents thereof known to those skilled in the art, and so forth.

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated by reference in full for the purpose of describing and disclosing methodologies that might be used in connection with the description herein. The publications are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is like, or identical with, a term that has been expressly defined in this disclosure, the definition of the term as expressly provided in this disclosure will control in all respects.

Antimicrobial resistance (AMR) remains a persistent problem in the treatment of bacterial infections. With resistance having been observed against nearly all major antibiotics, 700,000 annual deaths are currently attributable to AMR globally and is projected to increase to as high as 10 million by 2050 without major interventions. One strategy for managing AMR is the large-scale sequencing of infection isolates, which has yielded tens of thousands of publicly available genome sequences for each major bacterial pathogen and frequently paired with resistance metadata.

This wealth of data has enabled global analyses on the genetics of AMR, many of which employ machine learning (ML) to predict AMR phenotypes directly from genetic variations. Accurate AMR phenotype prediction models trained on thousands of genomes have been developed for many pathogens such as Escherichia coli, Klebsiella pneumoniae, Mycobacterium tuberculosis, Salmonella enterica, or multiple species, with numerous others developed from smaller datasets. However, many of these studies report a significant fraction of their models' predictive genetic features to have no relationship to known AMR mechanisms. This disconnect between statistically identified and mechanistically established genetic determinants of AMR highlights the current gap in knowledge in AMR genetics and remains a challenge for the real world adoption of ML-based systems for rapidly predicting AMR and informing treatment strategies.

Data-centric efforts to close this gap have focused on genome-wide association studies (GWAS), yielding tools for conducting the statistical tests underlying GWAS such as PLINK and GEMMA, with some specializing in microbial GWAS by rigorously addressing population structure such as DBGWAS and Pyseer. However, the predictive features identified when training ML models to predict AMR naturally provide another source of AMR gene candidates distinct from GWAS. Such analyses can leverage the extensive body of ML literature and tools to draw from a broader range of statistical and algorithmic frameworks than those previously used for GWAS, and as such, there is a growing effort towards developing ML workflows specifically aimed at mechanism discovery within AMR genetics and beyond. Recent ML-aided GWAS have identified genetic and metabolic mechanisms behind resistance in M. tuberculosis, and demonstrated ML's competitiveness compared to typical statistical testing at recovering known AMR genes for multiple pathogens. These successes demonstrate the potential for ML to carry out the role of GWAS for the ever-growing public collection of bacterial genome sequences and AMR data.

A machine learning analysis is presented herein which is tailored towards AMR gene discovery consisting of three components drawn from previous workflows: pangenome construction by sequence clustering to enumerate biologically interpretable genetic features, systematic annotation of known AMR genes among those features with RGI, and training of support vector machine (SVM) ensembles to learn relationships between all genetic features and a given AMR phenotype. This workflow was evaluated for both phenotype prediction accuracy and recovery of known AMR genes among predictive features across 12 pathogenic species spanning 127 species-drug combinations and 27,155 genomes. We find that this approach provides both a comprehensive overview of the distribution of known AMR genes and consistently yields ML models that both accurately predict AMR phenotype and outperform contemporary GWAS methods at recovering known AMR genes. Functional analysis of strongly predictive features yielded a set of 142 AMR gene candidates, of which two were experimentally confirmed to impact resistance in E. coli.

The exponential growth of publicly-available bacterial genome sequences and resistance metadata provides valuable opportunities for applying statistical methods to elucidate the genetics of AMR at a global scale. By applying workflows in pangenome construction, systematic AMR gene annotation, and machine learning to 27,155 genomes, 12 species, and 176,911 SIR phenotypes, the disclosure characterized the current interspecies distribution of known AMR genes and demonstrated the broad capability of ML to carry out GWAS analyses of AMR, surpassing contemporary statistical methods at recovering known AMR genes. From the most accurate ML models, 142 AMR genetic feature candidates were identified, two of which were experimentally verified to impact resistance in E. coli. Many of these results depend on the reliable recovery of rare biological features and events and were enabled by the scale of this analysis, operating on the largest internally-consistent AMR phenotype dataset known in the art.

First, an analysis of 6,332 known AMR genes revealed 925 (14.6%) genes to be present in multiple species, which tended to be plasmid-encoded over chromosomally-encoded in a function-dependent manner. This result is consistent with previous studies finding plasmids frequently responsible for cross-species and cross-genera transfer of AMR genes in clinical environments. However, AMR gene transfer is much rarer between species differing at higher phylogenetic ranks, with just 8 AMR genes observed in at least 10 genomes outside their main phylogenetic class. It has been suggested that transfer of AMR genes between unrelated species such as between gram-positive (GP) and gram-negative (GN) species is rare but possible, having been inferred for tetracycline resistance proteins, ermB, aph(3′), and observed for some beta-lactamases.

A case study of blaTEM revealed one variant, TEM-116, to be present in the GP species S. aureus and found among S. aureus strains with the highest levels of resistance to beta-lactams. TEM-116 was observed on three plasmids of which plasmid NZ_AJ437107.1 was observed in both GP and GN strains, suggesting a potential route of transfer. While blaTEM was only recently reported in S. aureus, the S. aureus genomes identified herein to harbor TEM-116 were isolated as early as 2009, suggesting that this transfer may be a much older phenomenon. Further analysis of isolation dates may enable the reconstruction of timelines for the spread of multispecies AMR genes, and help identify whether specific species act as reservoirs for enabling interspecies AMR gene transfer. Given these observations, the transfer of AMR genes between GP and GN strains should be treated as a potential significant contributor to the spread of resistance, and large-scale analyses will likely be necessary to continue capturing these rare events of AMR gene transfer between unrelated strains.

A ML workflow for identifying AMR-associated genetic features was developed by optimizing models for both phenotype prediction accuracy and biological relevance, with the latter quantified through a score based on the rankings of known AMR genes among a model's predictive features. Among cases with over 1000 SIRs and many known AMR genes, the GWAS score was found to generalize well to unseen known AMR genes, based on experiments randomly hiding known AMR genes from GWAS score calculation. Next, in a systematic analysis of four HPs, the optimal ensemble size was rarely found to exceed 50 estimators, much lower than previous works and suggesting that smaller, more computationally efficient models are sufficient at this scale. Feature subsampling was also confirmed to improve the recovery of known AMR genes without compromising accuracy in a majority of cases. However, HP optimization offered only modest improvements over using fixed HPs, in contrast to a previous GWAS analysis with neural networks on datasets of comparable scale which found HP optimization to significantly improve performance.

Applying this approach to 127 species-drug cases yielded models that were both accurate and reliably recovered known AMR genes. Compared with Pyseer and Fisher's exact test, SVM ensembles recovered nearly twice as many known AMR genes in total and near supersets of what could be recovered by the other methods. AMR genes recovered by multiple methods were concentrated among the top 3 features of the corresponding SVM ensemble, suggesting that only genes with the strongest statistical signals are reliably recovered by Pyseer or Fisher's exact test. Additionally, these results confirmed at a larger scale that accuracy is necessary but does not guarantee biological relevance. This is likely due to strong population structure resulting from the clonal nature of bacteria, which can lead to significant correlations between causal and hitchhiker mutations that are difficult to distinguish using statistical approaches. ML approaches can supplement AMR gene recovery without requiring the often computationally expensive task of defining population structure in advance. These results suggest that even small SVM ensembles with limited HP tuning can reliably carry out GWAS analyses for AMR genetics.

Analysis of the most accurate models yielded 142 AMR gene candidates, two of which were selected for experimental validation: amino acid transporter CycA vs. quinolone resistance, and fumarate reductase subunit FrdD vs. beta-lactam resistance. Testing the effects of cycA KO vs. WT in E. coli in various environments, it was found the cycA KO confers modest but significant, conditional resistance against CIP, specifically in minimal media supplemented with the CycA substrate D-serine. One possible explanation of this result involves the 1) toxicity of D-serine, which may be mitigated by reducing uptake through loss of CycA or competitive inhibition by other CycA substrates in rich media, and 2) the SOS response, which is triggered to differing extents by both D-serine and fluoroquinolones and may result in an SOS response induction no longer optimal to either stress and ultimately greater CIP susceptibility. As neither CycA nor D-serine directly influence CIP's mechanism of action yet ultimately impact growth under CIP exposure, these results provide an example of separate environmental stresses and related genes measurably influencing AMR. Further investigation into D-serine may continue to shed light onto clinically relevant conditional resistance, as D-serine has also been shown to sensitize S. aureus to various beta-lactams and concentrations of D-serine similar to those tested here may be encountered in various host environments. CycA substrate glycine has also been shown to sensitize serum-resistant E. coli but not ΔcycA mutants, suggesting that CycA plays a role in multiple cases of environment-dependent resistance.

Similar experiments on the effect of the frdD V111D substitution on beta-lactam resistance revealed that resistance was conferred only in the presence of beta-lactamase encoding ampC, and comparing synonymous codons found this was likely due to overexpression of ampC as its promoter overlaps with the frdD open reading frame. This substitution was previously associated with ampicillin resistance in E. coli induction experiments but not discussed in relation to ampC, while a similar synonymous mutation yielding V117V was previously shown to be enriched in amoxicillin resistant E. coli and attributed to ampC overexpression. Given the prevalence of overlapping genes in bacterial genomes, future GWAS analyses that identify candidate genes associated with a phenotype will benefit from incorporating the genetic context of such genes into their analysis.

Overall, combining pangenomics, systematic gene annotation, and ML provides a workflow for efficiently uncovering patterns of known and candidate AMR genes at the scale of 10,000s of genomes with potentially greater reliability than contemporary statistical methods such as Pyseer or Fisher's exact test. The flexibility of ML provides numerous opportunities to continue improving this workflow, such as the direct integration of the known AMR gene recovery into the loss function, use of different model architectures beyond SVMs, input of additional genetic feature types such as SNPs, or benchmarking against other phenotypes beyond AMR. As the number of genome sequences continues to grow, periodic updates to this analysis with improved techniques will be able to capitalize on the benefits of scale and steadily deepen the understanding of AMR across the phylogenetic tree.

The following examples are meant to further illustrate the invention and certain embodiments, but not limit the broader disclosure or the appended claims.

### EXAMPLES

Genome selection. An initial set of genomes was taken from the PATRIC database RELEASE_NOTES (ftp.patricbrc.org/RELEASE_NOTES/, 2021-07-21) and filtered by taxon ID to 12 species among the ESKAPEE pathogens or WHO global priority pathogens released in 2017. For each species, genomes were filtered to those meeting four criteria: 1) genome status is “WGS” or “Complete”, 2) number of contigs is within 2.5 times the median number of contigs across all assemblies for that species, 3) number of annotated CDSs is within 3 standard deviations of the mean, and 4) total genome length is within 3 standard deviations of the mean. Genomes were further filtered for those having EvalCon fine consistency of at least 87%. Drugs with at least 100 experimental antimicrobial (AMR) measurements were identified per species, and genomes were filtered to those with data for at least one of those drugs. Genome MLST subtypes were annotated using mlst v2.18.0 ([https://]github.com/tseemann/mlst) (note that brackets in the foregoing are to prevent active hyperlinks), and BioProject IDs were taken directly from metadata available on PATRIC.

Pangenome construction and genetic feature identification. For each species, genes (protein sequence clusters), alleles (protein sequence variants), and ORF-flanking sequence variants were identified using a sequencing clustering approach. All unique protein sequences across all genome assemblies (as annotated by PATRIC) were clustered using CD-HIT v4.6 with minimum identity 80% and minimum alignment length 80%. Each cluster was treated as a gene, cluster members as alleles, and the 300 bp upstream of the start codon of each occurrence of the gene as 5′ variants (and analogously the 300 bp downstream of stop codons as 3′ variants). 5′/3′ variants of a gene were identified by locating all occurrences of all alleles across all genome assemblies and extracting the 300 bp directly flanking those occurrences. Cases in which a 300 bp flanking region is interrupted by a contig break were ignored (for ML purposes, such genomes were treated as not having any specific 5′/3′ variant). A similar clustering analysis was conducted for non-coding features (annotated by PATRIC as “transcript”, “tRNA”, “rRNA”, or “misc binding”) to identify non-coding feature clusters and nucleotide sequence variants, using CD-HIT-EST v4.6 with the same parameters. The species-wide genetic variation covered by these six feature types was represented as a binary matrix based on presence/absence calls of each feature for each genome.

Processing antimicrobial resistance phenotypes and SIR phenotype inference from MICs. Species-drug cases with at least 100 experimental susceptible-intermediate-resistant (SIR) phenotypes or minimum inhibitory concentration (MIC) measurements among selected genomes were identified. For each pair, the most common SIR testing standard (i.e. CLSI, EUCAST) was identified from either the metadata or manual curation of BioProject accession IDs, referred to as the species-drug case's primary standard.

MIC values were mapped to SIRs by first filtering MICs for exact values mg/L values (opposed to bounded MICs) derived from one of the following laboratory typing methods: agar_dilution, agar_dilution_or_etest, bd_phoenix, bd_phoenix_and_etest, broth_microdilution, etest, mic, mic broth microdilution, liofilchem, sensititre, vitek_2. For each species-drug case, MIC-SIR mappings were generated for all MIC-SIR value pairs reported in at least three genomes under the primary standard. Ambiguous mappings (MIC value mapped to multiple SIRs) and inconsistent sets of mappings (instances where a susceptible MIC is greater than an intermediate or resistant MIC, or where a resistant MIC is less than an intermediate or susceptible MIC) were removed. A MIC-to-SIR inference scheme was developed as follows:
Exact MICs: Mapped directly to the corresponding SIR if possible.
Upper bounded and unmapped exact MICs: If MIC ≤largest MIC mapped to susceptible, it is mapped to susceptible.
Lower bounded and unmapped exact MICs: If MIC smallest MIC mapped to resistant, it is mapped to resistant.
Other unmapped exact MICs: If the MIC value is within the range of MICs mapped to intermediate, it is mapped to intermediate.

Reported and inferred SIRs were combined for subsequent analyses. For genomes with multiple conflicting SIRs for a single drug (i.e. measured by different methods), the most common SIR across all methods and inferences was selected, with directly reported SIRs breaking ties and perfect ties ignored. SIRs were binarized into susceptible and non-susceptible by converting “susceptible”, “susceptible-dose dependent”, and “non-resistant” to 0s, and “resistant”, “intermediate”, “non-susceptible” and “IS” to 1s for subsequent analyses.

Identification and classification of known AMR genes: All protein sequences for each species were annotated using RGI v5.2.0 with CARD ontology v3.1.327. To link AMR genes identified by RGI to specific drugs, a directed graph was constructed from the CARD ontology using ARO accession IDs as nodes and adding directed edge “U→” whenever:
U corresponds to a gene and U has the relationship “is_a” to V.
U corresponds to a drug and V has the relationship “is_a” to U.
U has the relationship “part_of”, “regulates”, “confers_resistance_to_antibiotic”, or “confers_resistance_to_drug_class” to V.
V has the relationship “has_part” to U.

A gene was labeled as conferring resistance to a drug if there exists a path from the gene's node to the drug's node in this graph. 23S rRNAs, 16S rRNAs, and 50S rRNAs were manually identified from PATRIC text annotations of noncoding features and were similarly linked to specific drugs using the graph, starting from nodes ARO:3000336, ARO:3003211, and ARO:3005003, respectively.

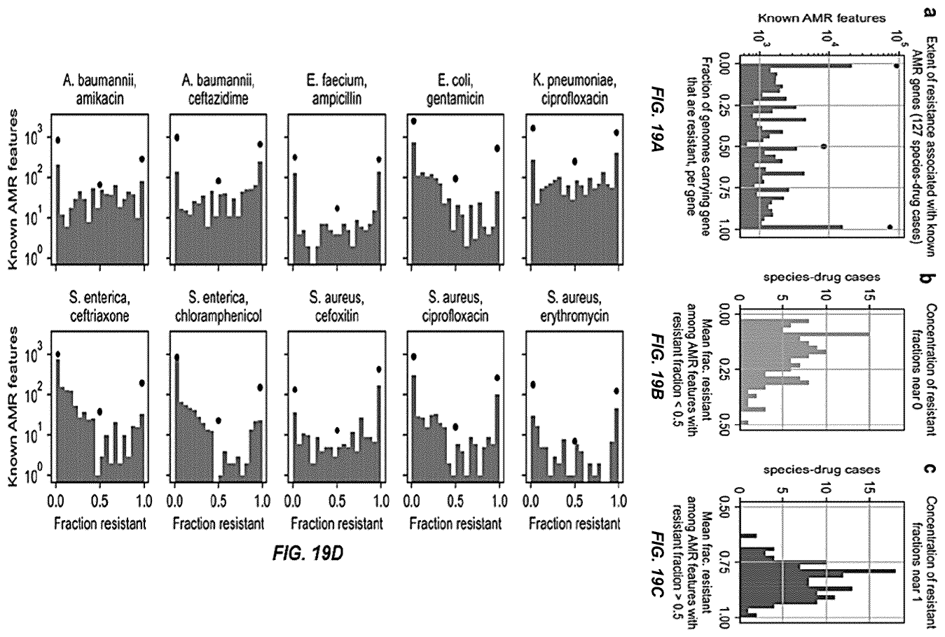

Additional drug-specific AMR genes were identified from PATRIC text annotations. Sequences with annotations containing a drug name or identical to the annotation of an RGI-identified AMR gene were identified and manually curated for probable known AMR genes. For machine learning purposes, all features associated with a gene cluster containing a sequence linked to resistance for a drug by RGI or PATRIC text annotation were treated as known AMR features. The distribution of AMR phenotypes for genomes carrying features annotated as known AMR is provided in FIG. 19.

AMR gene cross-species comparison, location prediction, and TEM beta-lactamase analysis. All alleles of identified AMR genes across all species were combined, de-duplicated, and re-clustered with the same CD-HIT parameters for cross-species analysis. Gene-level functional annotations for re-clustered AMR genes were inherited from corresponding allele-level annotations from RGI and PATRIC. AMR gene functional categories were assigned based on RGI annotations when available and PATRIC annotations otherwise, with categories occurring less than 50 times grouped as “other”.

All contigs from all assemblies were labeled as plasmid or chromosomal based on de novo predictions from PlasFlow v1.1 on default settings. Contigs were also mapped to known plasmids in PLSDB version 2021_06_23 using MASH v2.3 with minimum shared kmers 500/1000. For a given AMR gene cluster, each instance of each allele was assigned a location based on the PlasFlow prediction for the contig containing that instance (chromosome, plasmid, unassigned). The overall location of a gene or allele was assigned as 1) “chromosome” if >90% of instances were chromosomal, 2) “plasmid” if >90% of instances were plasmid, 3) “chromosome-leaning” if >50% of instances were chromosomal, 4) “plasmid-leaning” if >50% of instances were plasmid, and 5) “ambiguous” otherwise.

Complete TEM-family beta-lactamases (blaTEMs) were identified by filtering all AMR alleles for mention of “TEM” in the RGI or PATRIC annotation, and for length at least 272aa (95% length of TEM-1). Mutations were called from pairwise global alignment of each allele to TEM-1 using the Biopython Align module with scores match=1, mismatch=−3, open_gap_score=−5, and extend_gap_score=−2. N/C-terminal deletions were ignored. Known TEM variants were identified based on exact matches in the CARD database. Presence of specific blaTEM plasmids in individual genomes was determined by filtering the previous MASH results against PLSDB for mappings with MASH distance <0.025. For the S. aureus analysis, cefoxitin was identified as the only drug for which MIC data was available among blaTEM-carrying S. aureus genomes. Known AMR genes among S. aureus genomes with cefoxitin MIC data were identified from exact matches to entries related to beta-lactams in the CARD database.

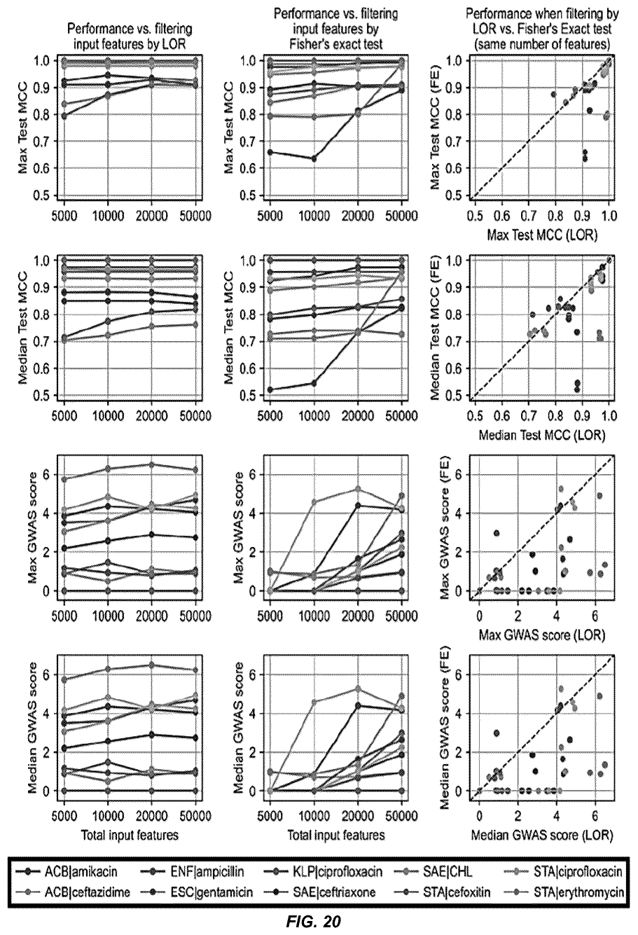

Implementation, evaluation, and hyperparameter optimization of SVM ensembles: For each species-drug case, SVM ensembles were trained to classify genomes as susceptible or non-susceptible (intermediate or resistant, referred to as “resistant”) based on the species' genetic feature presence/absence matrix. To accelerate training, feature count was reduced in three stages: 1) features present or missing in less than 3 genomes were removed, 2) perfectly correlated features were merged, and 3) remaining features were sorted by log odds ratio (LOR) for resistant genomes, and features with the 25,000 highest and 25,000 lowest LORs were retained (variations to this input filter were tested (as described more fully below; see also FIG. 20). SVM ensembles were implemented using scikit-learn v1.0.1 classes LinearSVC and BaggingClassifier, with square hinge loss (loss=‘squared_hinge’) weighted by class frequency (class_weight=‘balanced’) to address class imbalance issues and L1 regularization (penalty=‘l1’) to enforce sparsity in feature selection.

Model performance was evaluated in 5-fold cross validation experiments. Phenotype prediction accuracy was scored as the mean Matthews correlation coefficient (MCC) on the test set. Biological relevance was scored using the following equation:

$$GWASScore = {\sum }_{r ∈ known} 0 . {5}^{( r - 1 ) / 10} \qquad ( Eq . 1 )$$

where r corresponds to the ranks of known AMR features associated with the specific drug when sorted by feature importance, with r=1 corresponding to the highest feature importance. Feature importance was computed as the absolute value of the mean of the feature's coefficients across all SVMs in the ensemble with access to the feature (i.e. selected during feature subsampling). For ties, the average rank was assigned to all tied features.

Hyperparameter (HP) ranges for SVM ensembles were first evaluated on 10 test species-drug cases (Table 1). These were selected by first filtering for cases with substantial available data (at least 1000 SIRs and 50 known AMR genes), then sampling for even representation of drug classes and species phylogenetic classes. 256 HP combinations from four HPs were tested for each test case: number of estimators per ensemble, fraction of samples per estimator (with replacement), fraction of features per estimator (without replacement), and the SVM regularization term C, corresponding to parameters ‘n estimators’, ‘max_samples’, and ‘max features’ in BaggingClassifier and ‘C’ in LinearSVC, respectively. For each test case, the highest MCC (mean test set MCC from 5-fold CV) and GWAS scores were computed across models for all HP combinations. The smallest subset of HP combinations was identified such that the best scores in the subset were within 90% of the best scores across all combinations, across all test cases Table 2.

TABLE 1
Representative species-drug cases for hyperparameter testing. Columns “n”, “% Sus.”
and “AMR genes” refer to the number of genomes with AMR data, the fraction
of those genomes that are susceptible, and the number of known AMR genes identified
that are associated with the drug for that species, respectively

| | | | | | % | AMR |
|---|---|---|---|---|---|---|
| Species | Species Class | Drug | Drug Class | n | Sus. | genes |
| A. baumannii | γ-proteobacteria | amikacin | aminoglycoside | 924 | 49.1 | 150 |
| A. baumannii | γ-proteobacteria | ceftazidime | beta-lactam | 960 | 15.3 | 151 |
| E. coli | γ-proteobacteria | gentamicin | aminoglycoside | 2447 | 86.6 | 263 |
| K. pneumoniae | γ-proteobacteria | ciprofloxacin | quinolone | 2089 | 19.1 | 227 |
| S. enterica | γ-proteobacteria | ceftriaxone | beta-lactam | 2259 | 84.2 | 154 |
| S. enterica | γ-proteobacteria | chloramphenicol | other | 2348 | 82.4 | 116 |
| E. faecium | Bacilli | ampicillin | beta-lactam | 1432 | 14.2 | 81 |
| S. aureus | Bacilli | cefoxitin | beta-lactam | 1041 | 23.5 | 68 |
| S. aureus | Bacilli | ciprofloxacin | quinolone | 1725 | 62.2 | 42 |
| S. aureus | Bacilli | erythromycin | other | 2014 | 71.7 | 44 |

TABLE 2
SVM hyperparameter ranges used during optimization. Larger initial ranges
were used for evaluating representative species-drug cases, from which reduced
ranges were derived for evaluating against all species-drug cases.

| Hyperparameter | Initial range | Reduced range |
|---|---|---|
| Number of estimators | 25, 50, 100, 200 | 25, 50 |
| Fraction of samples per estimator | 25%, 50%, 75%, 100% | 50%, 75%, 100% |
| Fraction of features per estimator | 25%, 50%, 75%, 100% | 25%, 50%, 75%, 100% |
| C (SVM regularization term) | 0.1, 1, 10, 100 | 0.1, 1, 10 |
| Unique combinations | 256 | 72 |

SVM ensembles were trained for each HP combination in the reduced set across 127 species-drug cases with at least 100 SIRs, 10 known AMR genes, and minority phenotype frequency >5%. For each case, the optimal HP set was selected by ranking all HP combinations by either MCC or GWAS score, taking the average of the two ranks as the HP set's overall rank.

Comparison between SVM ensembles, Pyseer, and Fisher's exact test at recovering known AMR genes. For each of the 127 species-drug cases tested, the top 10, 20, and 50 genetic features associated with the AMR phenotype were identified for each GWAS approach. For SVM ensembles, features were sorted by feature importance as previously defined. For Fisher's exact test, the test was applied between each genetic feature and the binary AMR phenotype (susceptible/non-susceptible), and features were sorted by p-value. For Pyseer, distances were first computed between each pair of genomes using MASH v2.3 with default parameters. Pyseer v1.3.10 was then run using binary AMR phenotypes for the—phenotype parameter, genetic feature presence/absence calls for the—pres parameter, MASH distances for the—distances parameter, and all other parameters default, and features were sorted by population structure-adjusted p-value “lrt-pvalue”.

Identification of candidate antimicrobial resistance determinants. The models for each of the 127 species-drug cases were filtered down to those achieving mean test MCC >0.8 during 5-fold cross validation. From each remaining model, the top 10 features by feature weight absolute value were identified, and filtered for those that 1) were not already known AMR genes, 2) occurred in at least 10 genomes with SIR data for the corresponding drug, and 3) had positive feature weight and LOR for resistance. Remaining feature-drug pairs were tested for whether the feature was significantly associated with resistance, applying Fisher's exact test for SIRs and Brunner-Munzel tests for MICs. Tests were applied for the specific drug and drugs of the same class for which at least 5 SIRs (for Fisher's exact) or 5 MICs (for Brunner-Munzel) were available, and significance was determined at FWER <0.05 with Bonferroni correction (2008 Fisher's exact tests and 1393 Brunner-Munzel tests were conducted, with significance thresholds of p<2.5*10⁻⁵ and p<3.6*10⁻⁵, respectively). To evaluate co-occurrence with known AMR genes, AMR features found in predominantly in resistant strains were identified for each drug, defined as those occurring in at least 5 genomes of which at least 90% are resistant. Each candidate feature-drug pair was assigned a score based on the sum of 1) number of drugs with significant association based on SIR data, 2) number of drugs with significant association based on MIC, and 3) number of drugs for which at least one resistant genome with the feature does not also have any AMR features found predominantly in resistant strains.

The top 10 features by score for each species-drug class pair were identified, yielding 142 candidates which were categorized by function as annotated by PATRIC and additionally by eggNOG-emapper v2.1.6-43. Genes that were poorly annotated, related to mobile genetic elements (transposases, insertion elements, phage elements, integrases, plasmid maintenance), or known to be associated with a specific AMR mechanism for an unrelated drug class were their own categories, and the remaining genes were categorized as well-characterized candidates. For perfectly correlated features, coding features were selected over noncoding features. For variant-level features, mutations were determined against the most common variant of the parent gene cluster using the Biopython Align module. Interpretation of E. coli candidates was conducted using reference genomes U00096.3 [see, world wide web at ncbi.nlm.nih.gov/nuccore/U00096.3] for K-12 MG1655 and NZ_CP009273.1 [https://www.ncbi.nlm.nih.gov/nuccore/NZ_CP009273.1] for BW25113.

Generation of frdD and cycA E. coli mutants. E. coli BW25113 knockout mutants ΔcycA and ΔampC were taken from the Keio collection. The frdD mutations referred to in this study were introduced into both BW25113 and ΔampC using a Cas9-assisted Lambda Red homologous recombination method. Golden gate assembly was first used to construct a plasmid vector harboring both Cas9 and lambda red recombinase genes under the control of an L-arabinose inducible promoter, a single guide RNA sequence, and a donor fragment generated by PCR which contained the desired mutation and around 200 bp flanking both sides of the Cas9 target cut site as directed by the guide RNA. After allowing the transformed cells to recover for 2 hours at 30° C., L-arabinose was added to the media and the cells were allowed to grow for 3-5 hours at which time a portion of the culture was plated. Single colonies were screened using ARMS PCR and amplicons spanning the mutation site, generated with primers annealing to the genome upstream and downstream of the sequence of the donor fragment contained in the plasmid, were confirmed with Sanger sequencing. Confirmed isolates were cured of the plasmid by growth at 37° C. Both of the frdD mutations that were introduced in this study fell within a guide RNA target sequence. Because Cas9 has a tolerance for some single base mismatches in the guide RNA, a second mismatch was engineered into the guide RNA so that the guide RNA had two mismatches with respect to the successfully mutated target sequence and only one tolerated mismatch with respect to the starting strain. In one case, an intermediary strain was first constructed in which all of the codons falling within the guide RNA target sequence were switched to synonymous ones maximizing the base changes. A second round of Cas9-assisted Lambda Red homologous recombination was then used to restore those codons to their original sequences and introduce the desired mutation at the same time.

Cell growth conditions and measurements. Two media were used for both cycA and frdD experiments: 1) Mueller-Hinton Broth (Sigma-Aldrich, SKU: 70192-500G) supplemented with 49 mM MgCl₂ and 69 mM CaCl₂, and 2) M9 minimal medium (47.8 mM Na₂HPO₄, 22 mM KH₂PO₄, 8.6 mM NaCl, 18.7 mM NH₄Cl, 2 mM MgSO₄, 0.1 mM CaCl₂) supplemented with 2 g/L glucose. For cycA experiments, media were also supplemented with either 10 mM glycine, D-serine, D-alanine, L-alanine, DL-alanine (50:50 mixture of D- and L-alanine) or nothing, for a total of 12 possible supplemented media.

Cell densities for strain-media-antibiotic combinations were measured in biological triplicates as follows: Fresh culture samples were prepared (OD₆₀₀=~0.05) in each relevant media. Sample solutions were loaded to Costar flat-bottom 96-well plates (Corning, catalog no. 3370), with antibiotics added to varying concentrations (8, 16, 31, 62, or 125 μg/L ciprofloxacin for cycA experiments, and 0.25, 0.5, 1, 2, 4, or 8 mg/L ampicillin for frdD experiments). Plates were incubated in a microplate reader (Tecan Infinite200 PRO) with shaking at 37° C., and ODs were read every 15 minutes. Maximum cell density for each condition and replicate was calculated as the maximum OD₆₀₀ over 12 hours after inoculation minus the minimum OD₆₀₀ observed for the corresponding media without inoculum. Significant differences in cell density between pairs of strains or conditions was determined with Welch t-tests (FDR <0.05, Benjamini-Hochberg correction).

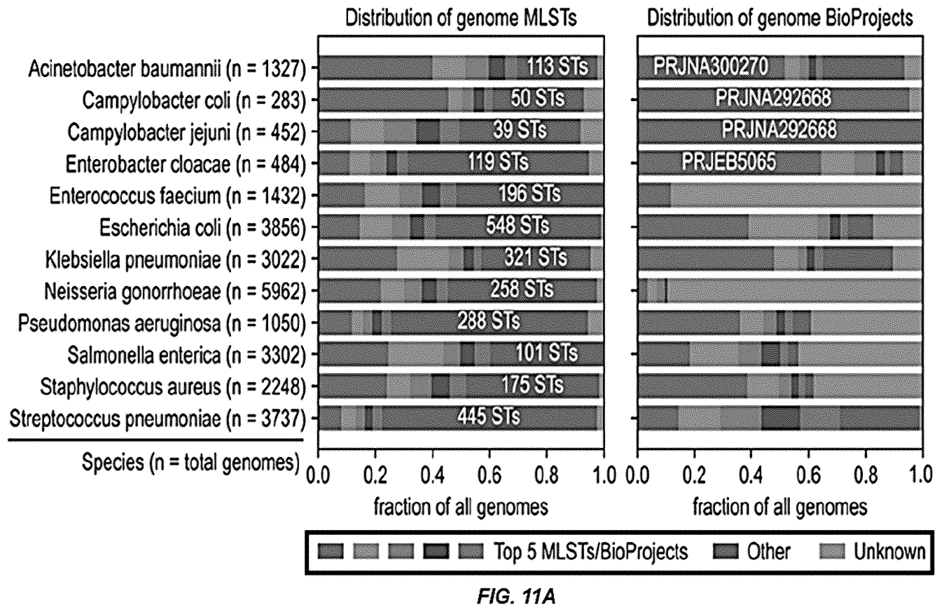

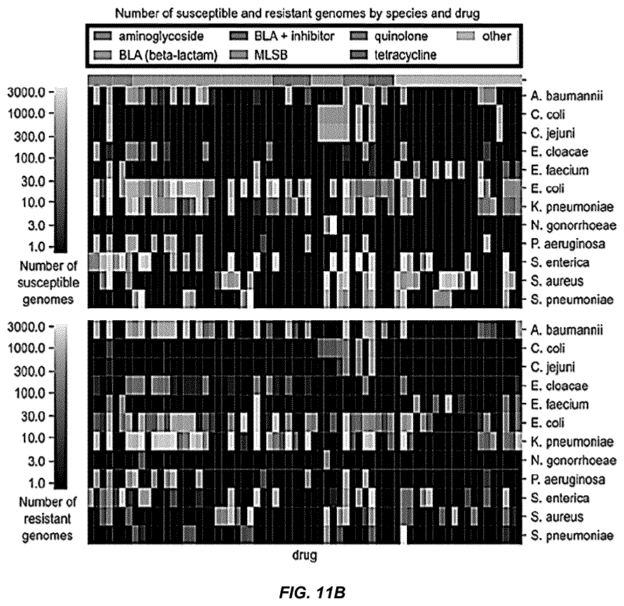

Diversity of selected genomes. The overall diversity of the genomes selected for this study was evaluated by examining the distribution of MLST subtypes (from [https://]github.com/tseemann/mlst) and BioProject IDs (from PATRIC) for each genome, and the number of susceptible/resistant genomes per drug (FIG. 11). MLST distributions suggest that the genome collection for each species is genetically diverse, with the most common subtype per species never exceeding 50% of all genomes and at least 44 subtypes represented per species. BioProject ID distributions suggest that a majority of the genome collections also represent a wide range of studies, though the smallest collections were dominated by individual studies (C. coli and C. jejuni genomes originated almost entirely from PRJNA292668, E. cloacae genomes were majority from PRJEB5065) and genomes for E. faecium and N. gonorrhoeae had poor coverage regarding study of origin. Finally, genome counts by resistance suggest that both susceptible and resistant strains across a wide range of drug classes are present for most species, with only the N. gonorrhoeae collection potentially being limited due to AMR data being available for only two drugs.

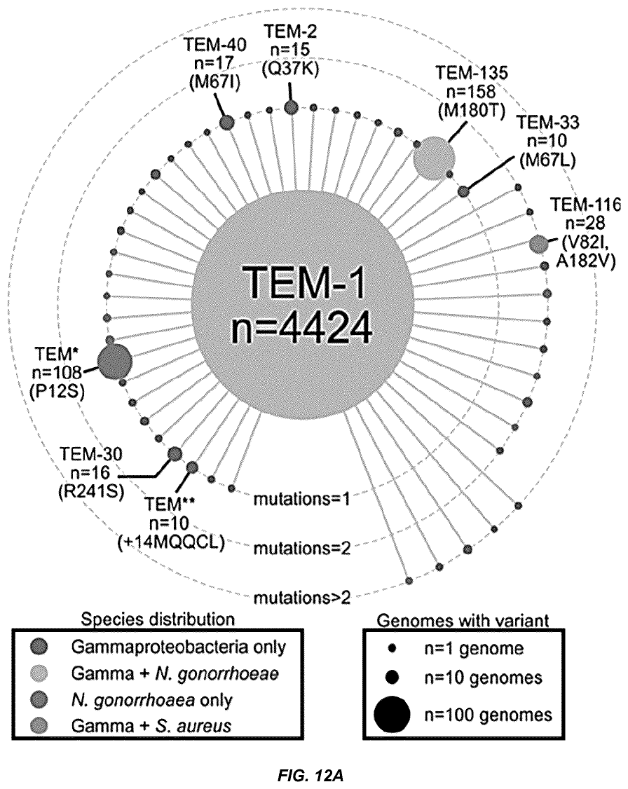

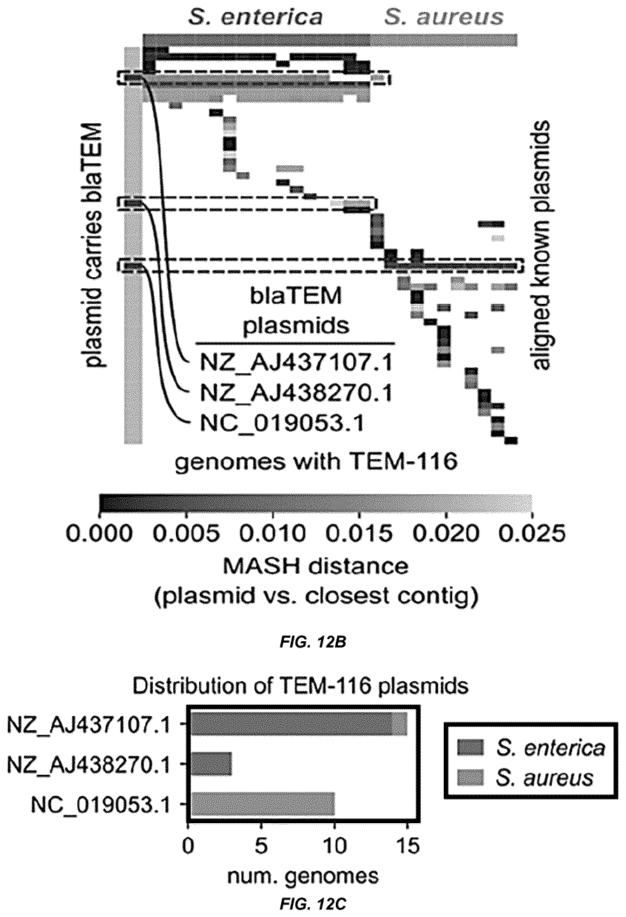

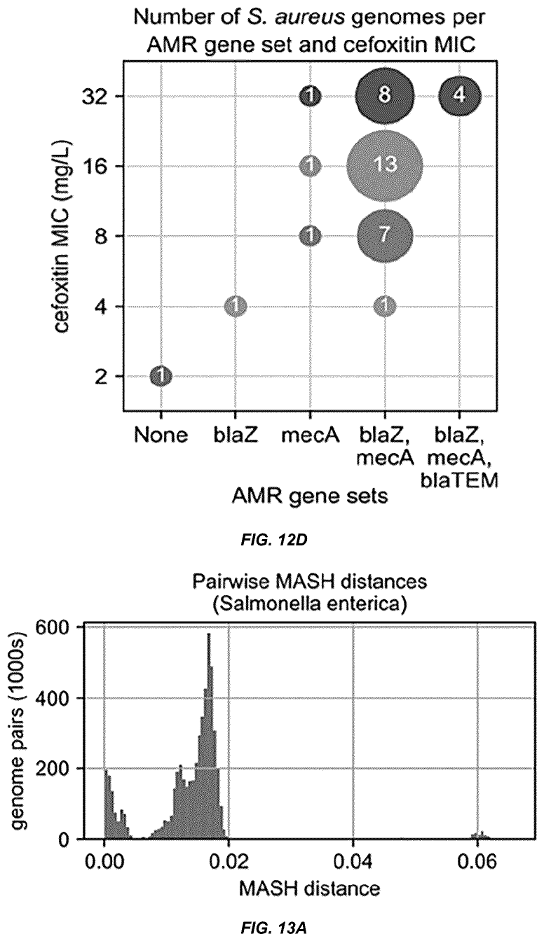

Observation of TEM-family beta-lactamases in both gram-positive and gram-negative strains. Given the prevalence of TEM family beta-lactamases (blaTEMs) among the few AMR genes spanning multiple phylogenetic classes, all observed blaTEM alleles were mapped to known blaTEM alleles based on RGI annotations, focusing on the 51 complete alleles defined as those with length within 5% of the TEM-1 allele. Complete blaTEMs were observed in 4,861 genomes spanning 8 species and were dominated by the TEM-1 allele occurring in 4,424 genomes (FIG. 12A, Table 3). All but five alleles were within two mutations of TEM-1, and only 9 alleles (including TEM-1) were observed in at least 10 genomes. Individual alleles were largely specific to phylogenetic class with 38 alleles limited to Gammaproteobacteria and 10 alleles limited to N. gonorrhoeae, compared to two alleles observed in both (TEM-1 and TEM-135). Just one allele was observed in gram-positive strains, TEM-116 (substitutions V82I and A182V relative to TEM-1), occurring in 11 S. aureus strains and 17 S. enterica strains. Contigs from genome assemblies harboring TEM-116 were mapped to known plasmids on PLSDB1 using MASH2, and the 28 instances of TEM-116 were predicted to be located on one of three plasmids: NZ_AJ437107.1 (14 S. enterica, 1 S. aureus), NZ_AJ438270.1 (3. S. enterica), and NC_019053.1 (10 S. aureus) (FIG. 12B-C).

TABLE 3
Distribution of the most frequently observed TEM-family beta-lactamases by
species. Mutations are shown relative to the TEM-1 variant. Variants are
ordered by total count. Species are abbreviated as A.
baumannii (AcB), E. cloacae
(EnC), E. coli (EsC), K.
pneumoniae (KlP), N. gonorrhoeae
(NeG), P. aeruginosa (PsA) , S. enterica
(SaE), and S. aureus (StA).

| | Number of genomes with variant by species | | | | | | | | | |
|---|---|---|---|---|---|---|---|---|---|---|
| Mutations | Variant | AcB | EnC | EsC | KlP | NeG | PsA | SaE | StA | Total |
| — | TEM-1 | 414 | 171 | 1692 | 1179 | 235 | 1 | 732 | — | 4424 |
| M180T | TEM-135 | — | — | 8 | 1 | 147 | — | 2 | — | 158 |
| P12S | * | — | — | — | — | 108 | — | — | — | 108 |
| V82I, A182V | TEM-116 | — | — | — | — | — | — | 17 | 11 | 28 |
| M67I | TEM-40 | — | — | 16 | 1 | — | — | — | — | 17 |
| R241S | TEM-30 | — | — | 16 | — | — | — | — | — | 16 |
| Q37K | TEM-2 | — | 14 | — | 1 | — | — | — | — | 15 |
| M67L | TEM-33 | — | — | 7 | 1 | — | — | 2 | — | 10 |
| 14insMQQCL | * | — | — | — | — | — | — | 10 | — | 10 |

* No exact match in the CARD database

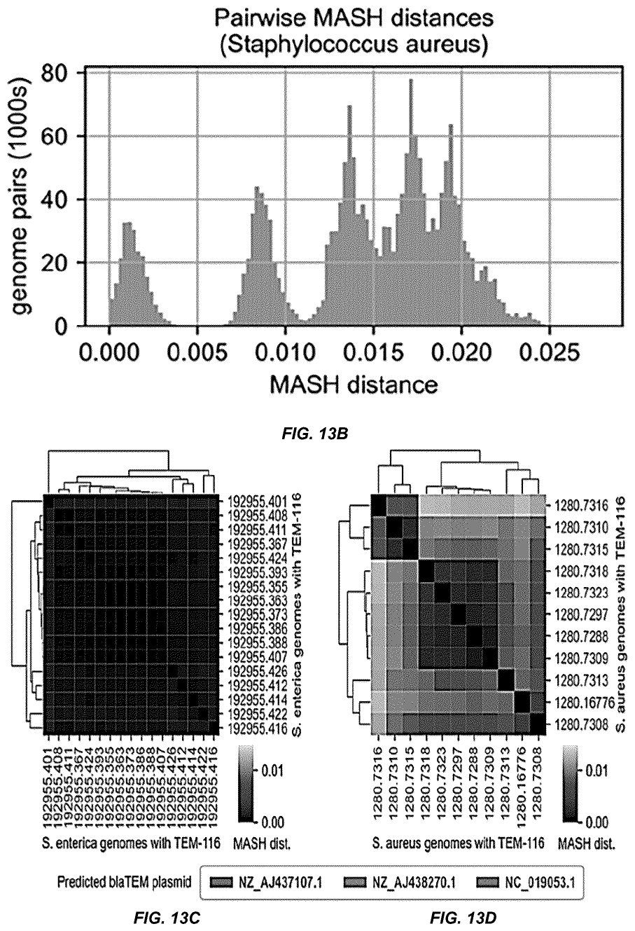

Pairwise MASH distances between these genomes were compared to those between all S. aureus and S. enterica genomes to assess sequence similarity and clonality (FIG. 13). The 17 S. enterica genomes with TEM-116 were highly similar with a median pairwise MASH distance of 0.0004 compared to a median of 0.0153 between all S. enterica genome pairs, consistent with the source study confirming all such genomes to be derived from a single clonal lineage of serotype Kentucky ST198, albeit from geographically diverse locations3. However, as the presence of the two TEM-116 plasmids in these genomes do not closely track their phylogeny, it is ambiguous as to whether the observed plasmid distribution was established earlier in the lineage and resulted from clonal dissemination, or was a result of more recent horizontal gene transfer events.

In contrast, the 11 S. aureus strains with TEM-116 were genetically diverse with a median pairwise MASH distance of 0.0080 compared to a median of 0.0151 between all S. aureus genome pairs. The genome 1280.16776, which is predicted to carry plasmid NZ_AJ437107.1 shared by some S. enterica genomes, is genetically distinct from the other TEM-116 carrying S. aureus genomes. Given the geographic prevalence of the S. enterica genomes carrying NZ_AJ437107.1 (annotated as isolated across 10 countries: Djibouti, Egypt, France, Indonesia, Israel, Kenya, Kuwait, Morocco, Tanzania, Vietnam), it is possible that the strain corresponding to 1280.16776 could have acquired this plasmid via horizontal gene transfer from a gram-negative species.

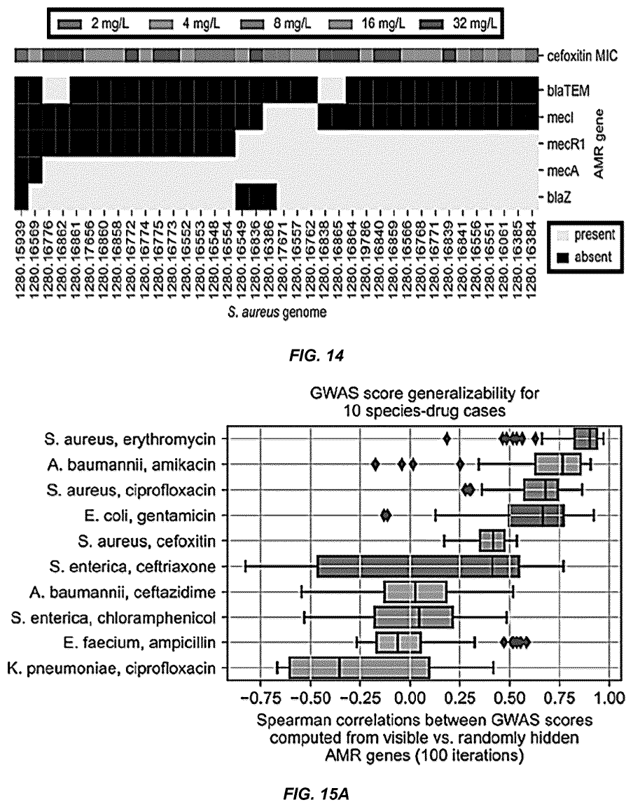

To assess the impact of blaTEM on beta-lactam resistance in S. aureus, the presence of blaTEM and other beta-lactam AMR genes was compared to cefoxitin MIC data, which was available for S. aureus genomes harboring blaTEM. While only four genomes with blaTEM also had MIC data, those genomes (which also carried mecA and blaZ) had significantly elevated MICs compared to the 29 genomes with just mecA and blaZ or mecA alone (p=0.0074, two-sided Mann-Whitney U-test, n₁=4, n₂=29), suggesting blaTEM may confer additional protection from beta-lactams in already resistant S. aureus (FIG. 12D, FIG. 14). These four strains were isolated from a study of the pork production chain in Shandong, China (BioProject: PRJNA433074), reflecting a geographically confined but genetically diverse sample set. The strains span two MLST subtypes (ST9, ST59), with three isolated from human workers (PATRIC IDs: 1280.16838, 1280.16865, 1280.16862) and one from a pig (1280.16776).

Association between features annotated as known AMR genes and observed resistance. For each known AMR feature across the 127 species-drug cases examined using the SVM workflow, the set of genomes carrying the feature was identified and the fraction of such genomes that are resistant to the corresponding drug was computed. The distribution of these resistance fractions was computed for each species-drug case individually and in aggregate, combining all known AMR features across all cases (FIG. 19). The distributions generally concentrated around resistance fractions of 0 or 1, which was quantified by computing the mean of resistance fractions for values either less than or greater than 0.5, for each species-drug case (FIG. 19B-C). These results suggest that features annotated as known AMR features are more likely than not to be related to resistance and are correctly labeled, i.e. features with resistance fractions closer to 1 are more likely to be resistance determinants, while those with fractions closer to 0 are more likely to be markers for susceptibility such as known fluoroquinolone-susceptible gyrA alleles.

Design of the GWAS score for biological relevance. The “GWAS score” was inspired by knowledge of follow-up analyses from past association studies. When hundreds of significant hits are observed, many GWAS and ML studies of AMR will focus on the top X features by effect size (i.e. top 3, 5, 10, 20). The GWAS score mimics this diminishing attention with respect to rank by giving exponentially diminishing marginal GWAS score per recovered known gene as rank increases, specifically, to diminish by half every 10 ranks.

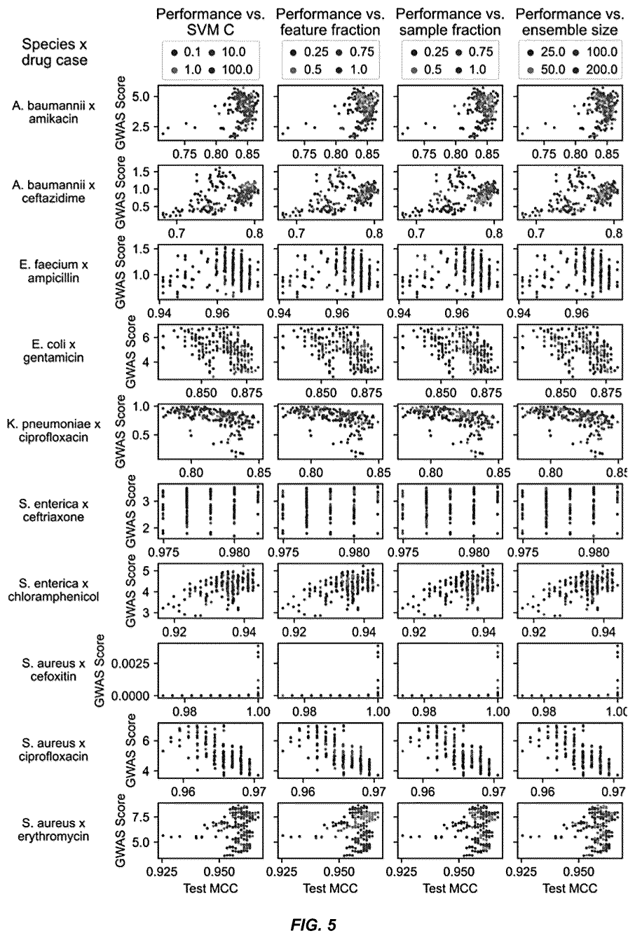

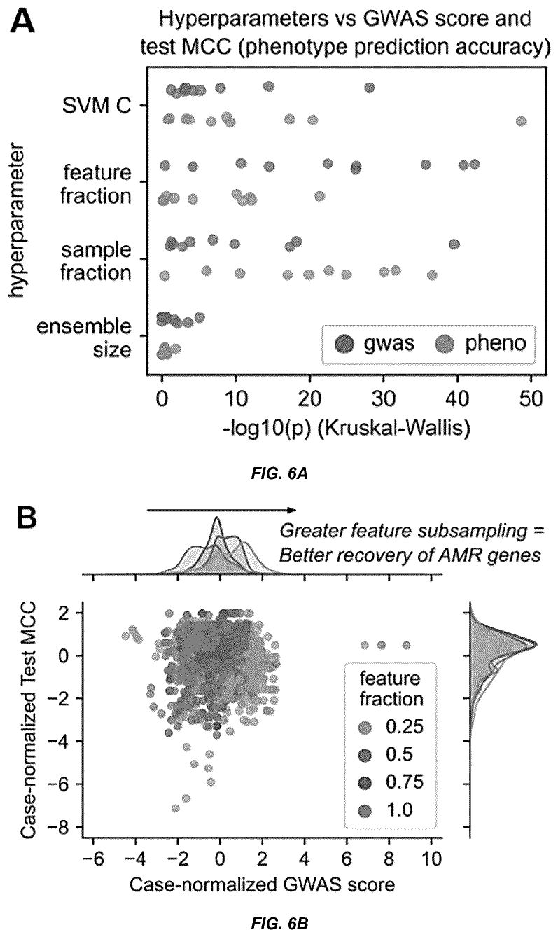

Support vector machine hyperparameter assessment on test cases. Ten test species-drug cases were selected among those with substantial data (at least 1000 SIRs and 50 known AMR genes), with balanced representation by drug class and species phylogenetic class (Table 1). Across these 10 cases and 256 hyperparameter (HP) combinations, it was found that ensemble size had little impact on either accuracy or biological relevance, while the other HPs significantly impacted both metrics: SVM regularization term C, fraction of samples per estimator, and fraction of features per estimator (Kruskal-Wallis test, n=256, FWER <0.05, Bonferroni correction, m=80 tests) (FIGS. 5 and 6A, Table 4). More aggressive feature subsampling (smaller fraction of features per estimator) also consistently improved on biological relevance without compromising accuracy. Applying two-sided Mann-Whitney U-tests to test for differences in GWAS scores from using feature fraction 25% over 50%, 50% over 75%, and 75% over 100% yielded p-values of 3.0*10⁻¹⁷, 6.0*10⁻³⁸, and 2.6*10⁻²⁹, respectively. Analogous tests for differences in test set MCC yielded 1.5*10⁻⁷, 0.03, and 0.27, respectively, of which only the test between 25% and 50% is significant (n₁=n₂=64, FWER <0.05, Bonferroni correction, m=6 tests). Finally, the smallest subset of HP combinations containing nearly-optimal models (within 90% of maximum MCC and GWAS scores) was identified for more efficient HP optimization of the remaining species-drug cases (Table 2).

TABLE 4
Negative log10 p-values for Kruskal-Wallis tests between SVM ensemble hyperparameters and
model performance. Performance metrics compared are phenotype prediction performance (mean
test set MCC during 5-fold cross validation) and recovery of known AMR genes among model
features (GWAS score). Each test was conducted across n = 256 hyperparameter combinations.

| | Hyperparameter vs. Test set MCC | | | | Hyperparameter vs. GWAS score | | | |
|---|---|---|---|---|---|---|---|---|
| | | feature | sample | ensemble | | feature | sample | ensemble |
| AMR Case | SVM C | fraction | fraction | size | SVM C | fraction | fraction | size |
| A. baumannii, amikacin | 1.009 | 12.123 | 6.016 | 0.75 | 1.967 | 26.219 | 17.297 | 0.711 |
| A. baumannii, ceftazidime | 17.258 | 0.187 | 24.916 | 0.58 | 3.212 | 0.402 | 39.543 | 0.011 |
| E. coli, gentamicin | 3.256 | 10.935 | 31.611 | 0.051 | 2.963 | 35.68 | 1.47 | 5.058 |
| E. faecium, ampicillin | 8.738 | 11.894 | 17.011 | 0.307 | 7.905 | 22.447 | 9.831 | 1.674 |
| K. pneumoniae, ciprofloxacin | 20.421 | 1.621 | 19.877 | 0.051 | 28.08 | 4.177 | 2.767 | 3.495 |
| S. aureus, cefoxitin | 48.629 | 0.117 | 0.353 | 0.001 | 14.449 | 10.696 | 1.244 | 0.001 |
| S. aureus, ciprofloxacin | 9.228 | 21.314 | 10.536 | 0.416 | 1.211 | 26.275 | 18.174 | 0.059 |
| S. aureus, erythromycin | 3.635 | 10.07 | 22.577 | 0.723 | 3.065 | 40.817 | 6.849 | 0.158 |
| S. enterica, ceftriaxone | 0.791 | 4.16 | 36.582 | 0.618 | 4.264 | 42.314 | 1.036 | 0.59 |
| S. enterica, chloramphenicol | 6.62 | 0.579 | 30.051 | 1.804 | 5.181 | 14.501 | 3.774 | 2.055 |

The impact of reducing the number of tested HP combinations was examined as follows. For each of the 10 test species-drug cases, the test set MCCs and GWAS scores across all folds (from 5-fold cross validation) were combined across all models resulting from tested HP combinations, then split into those associated with included HP combinations (n₁=72 HP combinations*5 folds=360) and those associated with excluded combinations (n₂=184 HP combinations*5 folds=920). Two-sided Mann-Whitney U-tests were applied to determine whether the MCCs or GWAS scores differed significantly between models with included or excluded HP combinations (Table 5). Significant differences were observed in 2/10 cases for MCCs and 5/10 cases for GWAS scores (n₁=360, n₂=920, FWER <0.05, Bonferroni correction, m=20 tests). In 2/2 significant MCC cases and 3/5 significant GWAS score cases, the subset had an equal or higher median score than the excluded combinations, suggesting that the reduction in the set of tested HP combinations only rarely reduces the maximum performance attainable.

TABLE 5
Statistically significant differences in model performance between selected and excluded
hyperparameter combinations for 10 species-drug cases. For each case, a two-sided
Mann-Whitney U-test was conducted to determine if the trained SVM model's test set
MCCs or GWAS scores, across all hyperparameter combinations (HPCs) and folds from
5-fold CV, differed significantly for HPCs included in the reduced HPC range than
for excluded HPCs. Starred cases are significant (n₁ = 72 HPCs * 5 folds =
360, n₂ = 184 HPCs * 5 folds = 920, FWER < 0.05, Bonferroni correction,
m = 20 tests).

| | Test set MCC | | | GWAS Score | | |
|---|---|---|---|---|---|---|
| | Included | Excluded | MW test | Included | Excluded | MW test |
| AMR Case | Median | Median | p-value | Median | Median | p-value |
| A. baumannii, amikacin | 0.83782 | 0.83815 | 0.23016 | 4.04567 | 4.07742 | 0.04913 |
| A. baumannii, ceftazidime | 0.76253 | 0.75629 | 0.00138* | 0.9075 | 0.59558 | 7.1*10⁻¹¹* |
| E. faecium, ampicillin | 0.95698 | 0.95698 | 0.12555 | 1.04808 | 1.07578 | 0.1873 |
| E. coli, gentamicin | 0.86522 | 0.86143 | 2.4*10⁻⁹* | 4.68256 | 5.1806 | 1.0*10⁻¹³* |
| K. pneumoniae, ciprofloxacin | 0.81835 | 0.81573 | 0.41189 | 0.88562 | 0.87238 | 0.34162 |
| S. enterica, ceftriaxone | 0.9751 | 0.9751 | 0.04874 | 2.74439 | 3.12598 | 1.0*10⁻¹⁷* |
| S. enterica, chloramphenicol | 0.93276 | 0.93276 | 0.12058 | 4.24349 | 4.28149 | 0.77218 |
| S. aureus, cefoxitin | 1.00000 | 1.00000 | 0.94434 | 0.00000 | 0.00000 | 4.1*10⁻¹³* |
| S. aureus, ciprofloxacin | 0.96917 | 0.96917 | 0.60248 | 4.94068 | 4.67576 | 0.00018* |
| S. aureus, erythromycin | 0.96318 | 0.9575 | 0.04671 | 6.23253 | 6.23332 | 0.37441 |

Variations on the preliminary feature filter. Variations to the approach of pre-filtering features by log odds ratio (LOR) were evaluated for their impact on downstream model performance. The existing filter of taking the top 50,000 features by LOR (specifically, the features with the 25,000 highest and lowest LORs) was compared to analogous filters taking the top 20,000, 10,000, or 5,000 features by LOR. These were also compared to filters taking the top 50,000, 20,000, 10,000 or 5,000 features by Fisher's exact test p-value when testing for enrichment in resistant genomes, for a total of eight possible feature filters. After applying each filter, SVM ensembles were trained to predict AMR phenotype across the 10 test species-drug cases and 256 HP combinations described in the previous section. The maximum and median test set MCC (from 5-fold cross validation) and GWAS score was computed for each species-drug case and under each filter (FIG. 20).

In most of the tested species-drug cases, the maximum and median MCC and GWAS scores are robust to the number of features provided, with a few exceptions under Fisher's exact test filters: Model MCCs for the E. coli-gentamicin and S. aureus-erythromycin cases and GWAS scores for the A. baumannii-amikacin, S. aureus-ciprofloxacin, and S. aureus-erythromycin cases increase with the number of features. When comparing equal size feature sets generated from filtering by either LOR or Fisher's exact test p-value, models derived from the LOR filter consistently performed better. These results suggest that (1) future applications of this ML workflow may be able to apply stricter preliminary feature filters to reduce the computational resources required for training with relatively little impact on performance, and (2) the LOR appears to more effectively identify features required for better performing models than the Fisher's exact test p-value, possibly due to its emphasis on effect size over significance.

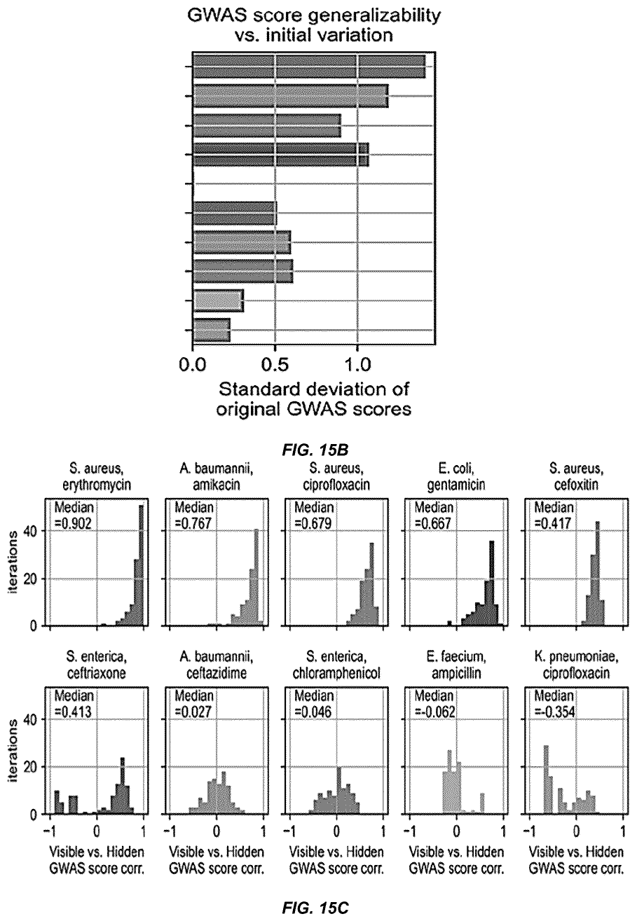

Generalizability of the GWAS score. To assess whether a model's GWAS score calculated from a fixed set of known AMR genes is representative of its capacity to recover other unseen AMR genes, the following experiment was conducted. For a given species-drug case, (1) half of all known AMR genes were randomly hidden, (2) GWAS scores were computed using either visible or hidden AMR genes across all 256 tested hyperparameter combinations, and (3) the Spearman correlation was computed between the two GWAS scores as a measure of generalizability. This experiment was conducted for n=100 random selections of hidden AMR genes for each of the 10 test species-drug cases. Overall, the results suggest that the GWAS score often generalizes well to hidden AMR genes, with a median Spearman correlation between GWAS scores from visible vs. hidden AMR genes exceeding 0.4 in 6/10 species-drug cases and 0.6 in 4/10 cases (FIG. 15A). The extent of generalizability was dependent on the initial level of GWAS score variation, i.e. the standard deviation of GWAS scores across all hyperparameter combinations with all AMR genes visible (FIG. 15B). Low initial variation in GWAS scores results in a less robust ranking of models by GWAS score, and consequently weaker correlations between GWAS scores calculated using different subsets of known AMR genes. Conversely, cases of high initial variation in GWAS score, i.e. those where HP optimization can meaningfully impact model performance, are more likely to have GWAS scores that are representative of the model's performance at recovering yet unknown AMR genes.

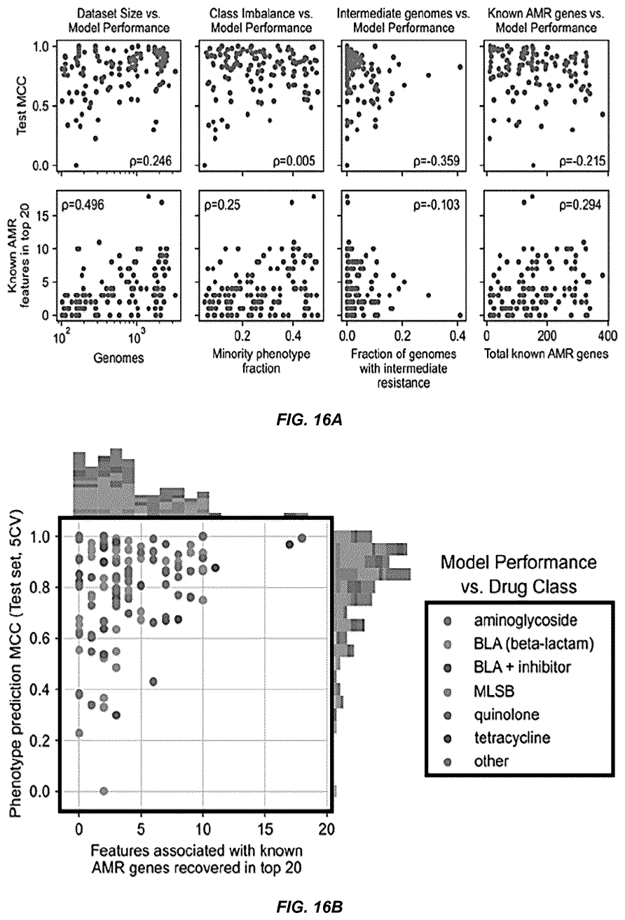

Effect of dataset parameters on model performance. The relationship between six dataset parameters and model performance was analyzed: species, drug class, dataset size (number of genomes), extent of class imbalance (minority phenotype fraction), fraction of genomes originally assigned the “intermediate” phenotype, and total number of known AMR genes annotated (FIG. 16, Table 6). Associations between these parameters and either the model's predictive performance (mean test set MCC from 5-fold cross validation) or known AMR gene recovery (number of AMR genes recovered among the top 20 features) were examined using Spearman R tests for quantitative parameters and Kruskal-Wallis tests for qualitative parameters, across n=127 species-drug cases. Of these, four tests were significant (FWER <0.05, Bonferroni correction, m=12 tests): Number of genomes vs. MCC, species vs. MCC, fraction of intermediate genomes vs. AMR gene recovery, and total known AMR genes vs. AMR gene recovery.

TABLE 6
Impact of input data properties on SVM ensemble performance. Impact
was assessed with Kruskal-Wallis tests for categorical properties
(species, drug class) and Spearman correlation for quantitative
properties (number of genomes, minority phenotype fraction, fraction
of genomes with the “intermediate” phenotype, total number
of known AMR genes). Spearman correlation tests are two-sided.
Each test was conducted over all 127 species-drug cases, and rows
are sorted by p-value. Starred cases are significant (n =
127, FWER < 0.05, Bonferroni correction, m = 12 tests).

| | | Statistical | |
|---|---|---|---|
| Performance Metric | Data Metric | Test | p-value |
| AMR genes in | num. genomes | Spearman-R | 3.1*10⁻⁹* |
| top 20 | | | |
| Test MCC in 5CV | species | Kruskal-Wallis | 7.6*10⁻⁷* |
| Test MCC in 5CV | intermediate fraction | Spearman-R | 0.00003* |
| AMR genes in | total known | Spearman-R | 0.00079* |
| top 20 | AMR genes | | |
| AMR genes in | minority fraction | Spearman-R | 0.00464 |
| top 20 | | | |
| Test MCC in 5CV | num. genomes | Spearman-R | 0.00537 |
| AMR genes in | species | Kruskal-Wallis | 0.00721 |
| top 20 | | | |
| Test MCC in 5CV | total known | Spearman-R | 0.01536 |
| | AMR genes | | |
| AMR genes in | intermediate fraction | Spearman-R | 0.25039 |
| top 20 | | | |
| Test MCC in 5CV | drug class | Kruskal-Wallis | 0.30250 |
| AMR genes in | drug class | Kruskal-Wallis | 0.48732 |
| top 20 | | | |
| Test MCC in 5CV | minority fraction | Spearman-R | 0.95993 |

Characterization of gyrA alleles associated with fluoroquinolone resistance recovered using support vector machine ensembles. All gyrA alleles among the top 50 features from models related to fluoroquinolone resistance were identified, totaling 30 alleles across 7 species. Mutations were called relative to the wildtype allele of the corresponding species, defined as the most commonly observed gyrA allele among genomes susceptible to at least one fluoroquinolone. All recovered gyrA alleles with positive feature weight for resistance had at least one known resistance-conferring mutation, covering the following substitutions: S81L in A. baumannii, T86I in C. coli and C. jejuni, S83L and D87N in E. coli, T83I in P. aeruginosa, and S83Y and D87* in S. enterica. All recovered gyrA alleles with negative feature weight for resistance had no such known resistance-conferring mutations. These results suggest that the gyrA alleles recovered by the SVM ensemble approach are consistent with current understanding of the gyrA mutational landscape with respect to fluoroquinolone resistance.

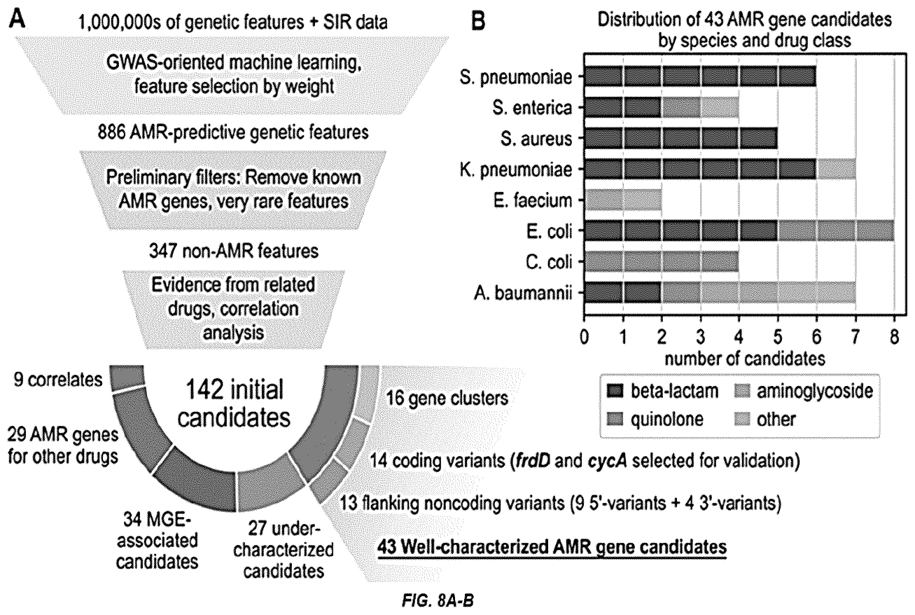

Selection of AMR gene candidates for experimental validation. AMR gene candidates were identified through a series of filters applied to the highest weighted features in the most accurate AMR models (FIG. 8A). The top 10 features by weight (including ties) across the 78 species-drug AMR models achieving test MCC >0.8 were combined to yield an initial set of 886 unique genetic features predictive of AMR. Of these, 610 were not directly associated with known AMR genes, 519 were also observed in at least 10 genomes, and 347 also had positive log odds ratios (LORs) for resistance against the drug for which the feature was predictive of resistance. Candidates in this intermediate set were then scored based on the sum of the following: (1) number of drugs in the same drug class for which the feature is significantly associated with resistance (applying Fisher's exact test to SIR data or applying Brunner-Munzel test to MIC data), and (2) number of drugs in the same drug class for which the feature appears in at least one resistant genome without any other known AMR genes (i.e., number of drugs for which this feature could explain previously unexplainable resistance). Finally, for each species-drug class pair, the top 10 features by this score were selected to yield the 142 AMR gene candidates (some pairs had fewer than 10 features remaining after these filters).

In the selection of features for experimental validation, the 142 candidates were further categorized by function and filtered down to 43 features that were (1) not poorly characterized, (2) not associated with mobile elements (transposases, insertion elements, phage elements, integrases, plasmid maintenance), (3) not AMR genes for unrelated drugs, and (4) not strongly correlated with a known AMR gene after manual inspection (FIG. 8B). Of these, six features referred to specific sequence variants in E. coli, a useful species to test experimentally. Two were excluded in order to focus on allele-type features and take advantage of the Keio knockout collection, and ultimately frdD and cycA were selected for experimental validation for their better functional and metabolic characterization compared to the other two options, sugE and yjfN.

Distribution of cycA, frdD, and ampC across E. coli genomes. Analysis of all 3,856 E. coli genomes in this study suggests that cycA, frdD, and ampC are core genes of E. coli, found in 3,823 (99.1%), 3,796 (98.4%), and 3,775 (97.8%) of genomes, respectively. No genomes had multiple copies of cycA, frdD, or ampC. 3,748 (97.2%) genomes had both frdD and ampC, of which all but three harbored frdD and ampC on the same contig. The distance between the frdD and ampC ORFs was highly consistent with a mean of 62.9 bp, standard deviation of 5.2 bp, and range of 30-192 bp. A vast majority of genomes (3,698) had a frdD-ampC distance of exactly 63 bp. This result suggests that a frdD V111D mutation will impact the ampC promoter in most E. coli strains.

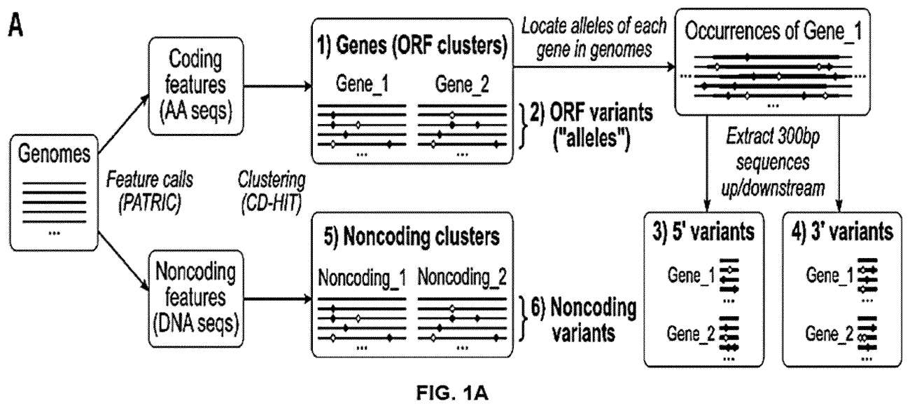

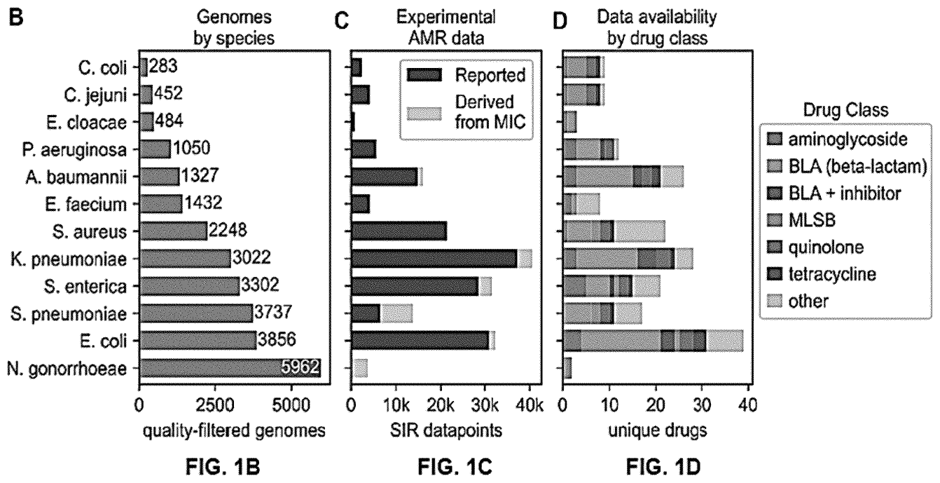

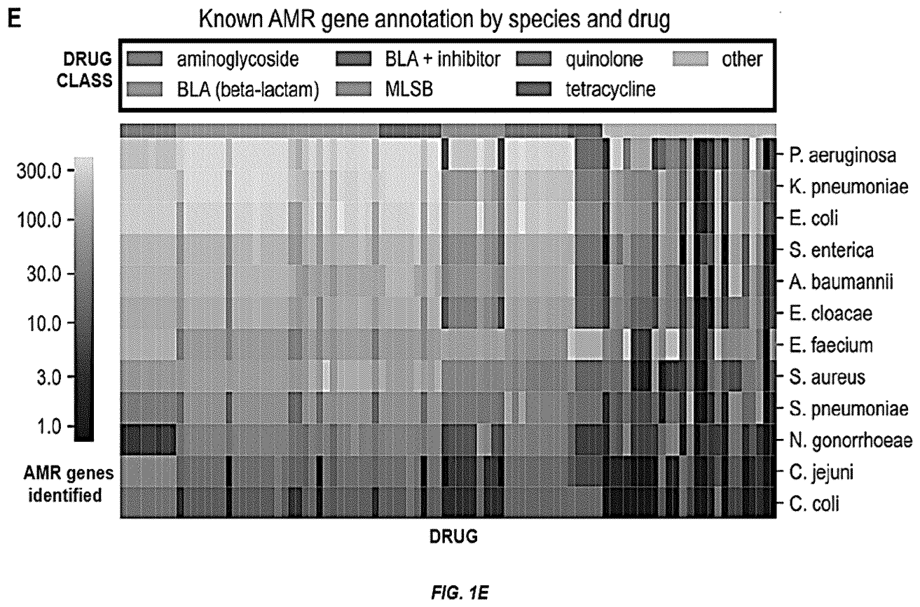

Assembly of twelve bacterial pathogen pangenomes and antimicrobial resistance data. A total of 27,155 genomes across 12 species were downloaded from the PATRIC database after filtering for assembly quality and availability of AMR data. Pangenomes were constructed and genetic features were enumerated for each species using a sequence clustering approach with CD-HIT v4.629, yielding six unique genetic feature types: 1) genes (protein sequence clusters), 2) alleles (protein sequence variants), 3) 5′ variants (300 bp ORF-flanking upstream variants), 4) 3′ variants (300 bp ORF-flanking downstream variants), 5) noncoding feature clusters, and 6) noncoding feature variants (see FIGS. 1A-B, and Table 7).

TABLE 7
Genetic feature counts by species.

| | | ORF-associated | | | | Non-ORF-associated | |
|---|---|---|---|---|---|---|---|
| | | | | 5′ flanking | 3′ flanking | noncoding | noncoding |
| species | genomes | genes | alleles | variants | variants | clusters | variants |
| A. baumannii | 1327 | 43888 | 239729 | 262420 | 275216 | 177 | 1725 |
| C. coli | 283 | 8299 | 44804 | 32011 | 31527 | 77 | 231 |
| C. jejuni | 452 | 9801 | 48710 | 35975 | 36276 | 82 | 381 |
| E. cloacae | 484 | 51969 | 325374 | 394577 | 406576 | 167 | 1716 |
| E. faecium | 1432 | 53518 | 174176 | 116209 | 113574 | 119 | 890 |
| E. coli | 3856 | 148886 | 1024333 | 1005186 | 1003017 | 435 | 5562 |
| K. pneumoniae | 3022 | 94147 | 562721 | 584114 | 596130 | 356 | 5010 |
| N. gonorrhoeae | 5962 | 18225 | 199856 | 122707 | 117320 | 74 | 693 |
| P. aeruginosa | 1050 | 67217 | 448882 | 558844 | 581936 | 136 | 903 |
| S. enterica | 3302 | 56315 | 262555 | 255349 | 261757 | 322 | 4932 |
| S. aureus | 2248 | 22649 | 210749 | 177169 | 173244 | 135 | 1826 |
| S. pneumoniae | 3737 | 36070 | 295716 | 269652 | 281790 | 110 | 1669 |

Experimentally derived susceptible-intermediate-resistant (SIR) phenotypes for the genomes were assembled from directly reported SIRs and SIRs inferred from minimum inhibitory concentrations (MICs). MIC breakpoints for SIR inference were determined from genomes with both SIR and MIC values for each species-drug-standard combination (i.e. CLSI, EUCAST) to yield internally-consistent SIR data. From 169,693 MICs, 22,772 SIR inferences across 93 species-drug cases were generated for genomes without SIR data. In total, 176,911 SIR phenotypes were assembled across 69 drugs, with 88.3% phenotypes from directly reported SIRs and 11.7% inferred from MICs (FIG. 1C-D), comprising the largest internally-consistent AMR dataset known at this time. The overall diversity of the combined genomic and AMR data was evaluated by analyzing the distribution of MLST subtypes, genome BioProject IDs, and susceptible/resistant genomes by drug (FIG. 11). Based on these evaluations, the genomes represented here are diverse with respect to subtype, study of origin, and resistance phenotypes, with cases of potentially lower coverage limited to species with AMR data for a smaller range of drugs (Neisseria gonorrhoeae) or fewer genomes available in total (Campylobacter coli, Campylobacter jejuni).

Known AMR genes were identified in each pangenome through direct annotation of alleles by RGI v5.2.0 with CARD ontology v3.1.3 and parsing PATRIC text annotations for drug-associated terms. 7,710 AMR genes were identified across all species, spanning 95,491 gene-drug mappings (FIG. 1E). The fewest number of AMR genes (71) were identified in Campylobacter coli and the most in Escherichia coli (1,533), with the greatest number of AMR genes identified for major drug classes such as beta-lactams, aminoglycosides, and quinolones.

Global analysis of known AMR genes reveals potential phylogenetic limitations on cross-species gene transfer.

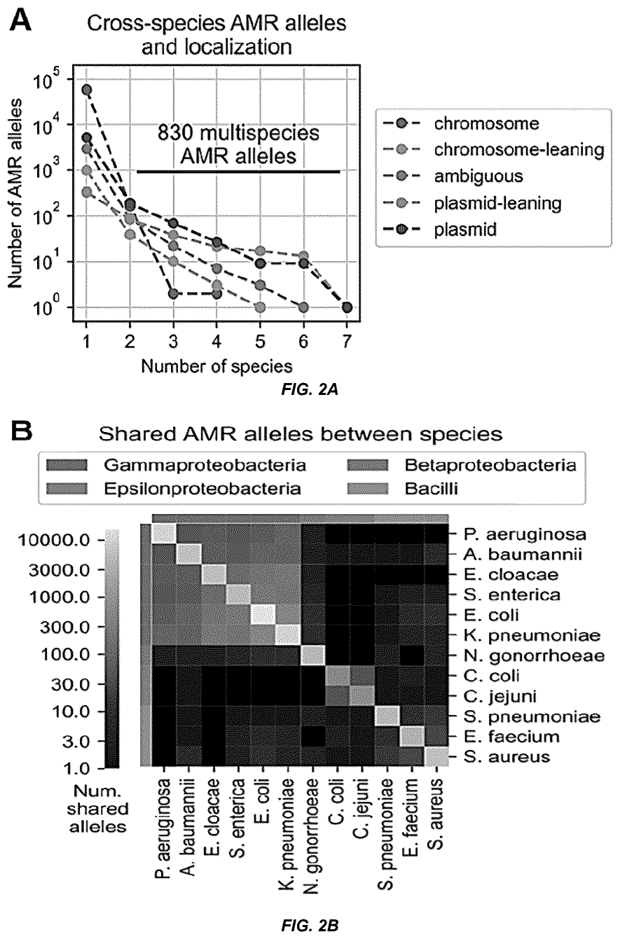

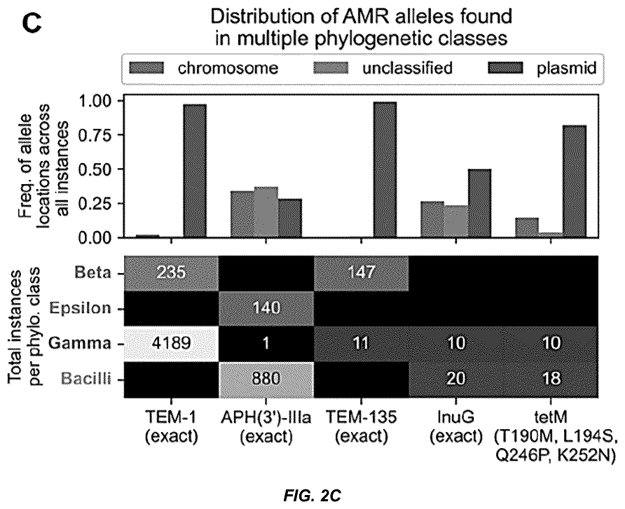

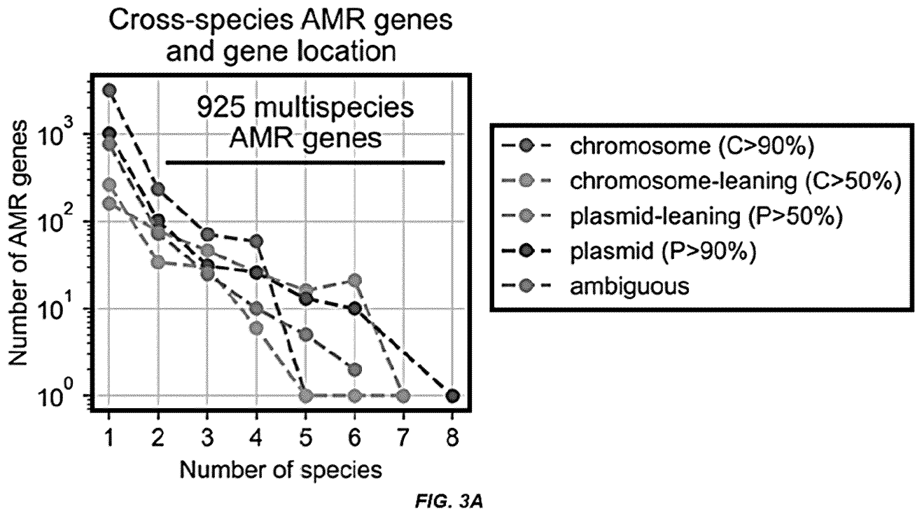

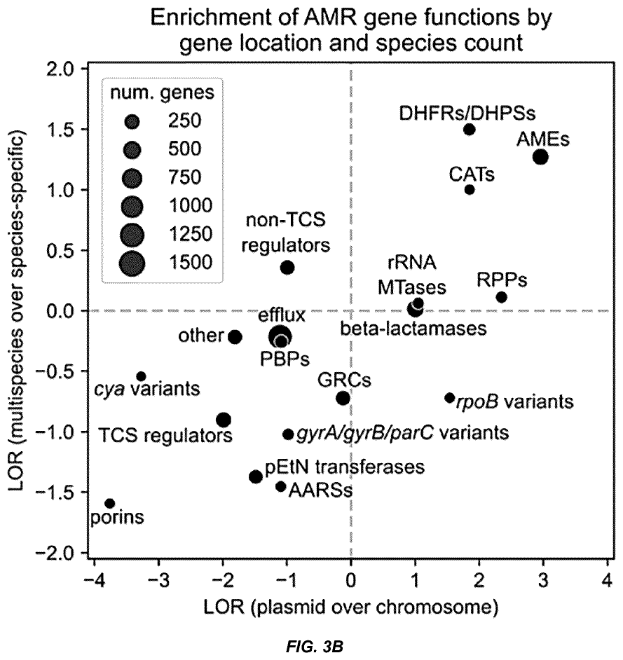

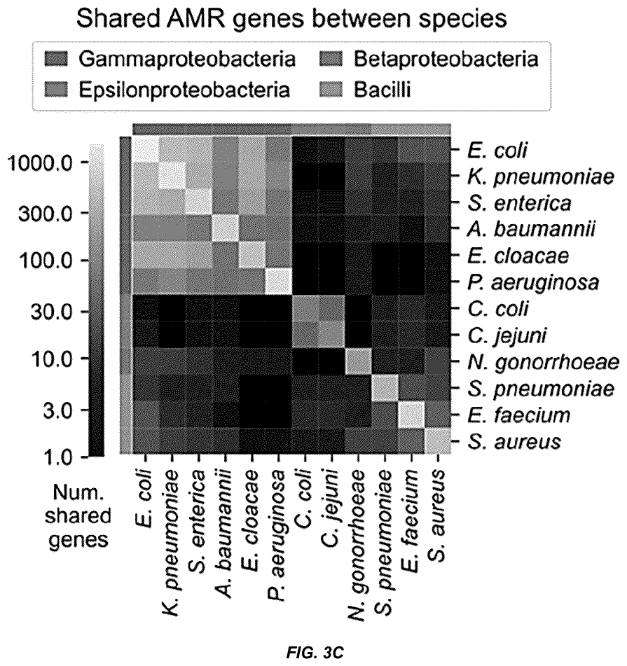

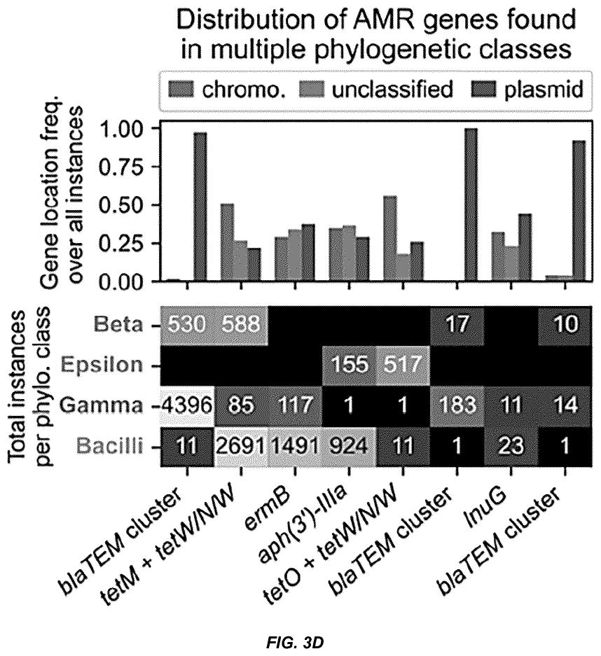

To examine the distribution of known AMR genes, all AMR gene alleles across all species were re-clustered with CD-HIT, yielding 6,332 unified AMR genes. Rates at which these genes were plasmid- or chromosomally-encoded were predicted by labeling contigs containing AMR genes as plasmid or chromosomal with PlasFlow. 925 AMR genes were observed in multiple species, with more broadly distributed genes having a greater tendency to be plasmid-encoded (95% of genes in >4 species were plasmid-encoded in a majority of occurrences) (FIG. 3A). Similarly, out of 68,324 unique AMR alleles, 830 were observed in multiple species and were primarily plasmid-encoded (FIG. 2A).

Compared against AMR gene categories, specific functions were significantly enriched among both plasmid-encoded (over chromosomal) and multispecies (over single species) AMR genes (FIG. 3B). Dihydrofolate reductases/dihydropteroate synthases and aminoglycoside modifying enzymes were significantly enriched by both measures (n=6,332 AMR genes, Fisher's exact test, FWER <0.05, Bonferroni correction, 36 tests), with log₂ odds ratios (LORs) for plasmid over chromosomal genes of 3.0 and 1.8, and LORs for multispecies over single species of 1.5 and 1.3, respectively. Other categories enriched in plasmid but not multispecies genes include chloramphenicol acetyltransferases, ribosomal protection proteins, rRNA methyltransferases, and beta-lactamases (plasmid LOR >1.0, multispecies LOR <1.0) (Table 8). Generally, multispecies AMR genes tended to be plasmid-encoded and vice versa, with the exception of rpoB variants (FIG. 3B). As rpoB is a highly conserved chromosomal bacterial gene31, this exception may be due to many rarely observed rpoB fragments on short contigs being misclassified as plasmid-encoded.

TABLE 8
Enrichment of AMR gene categories in multispecies over single species genes and plasmid
over chromosomal genes. For each comparison, “genes” is the total number of genes
in the category, “LOR” is log2 odds ratio, and “p-value” is two-sided Fisher's
exact test p-value. Starred values are significant (n = 6,332 AMR genes, FWER <
0.05, Bonferroni correction, m = 36 tests). Categories are sorted by LOR for plasmid
over chromosomal genes. Only categories with at least 50 genes were tested.

| | Multispecies over Single species | | | Plasmid over Chromosomal | | |
|---|---|---|---|---|---|---|
| AMR gene category | genes | LOR | p-value | genes | LOR | p-value |
| aminoglycoside modifying | 563 | 1.272 | 3.8*10⁻¹⁶* | 455 | 2.957 | 3.2*10⁻⁸⁹* |
| enzymes (AMEs) | | | | | | |
| ribosomal protection proteins | 154 | 0.113 | 0.72879 | 115 | 2.348 | 4.2*10⁻¹⁷* |
| chloramphenicol acetyltransferases | 91 | 1.001 | 0.00656 | 77 | 1.850 | 3.4*10⁻⁸* |
| (CATs) | | | | | | |
| dihydrofolate reductases (DHFRs), | 219 | 1.498 | 1.3*10⁻¹⁰* | 191 | 1.845 | 1.3*10⁻¹⁷* |
| dihydropteroate synthases (DHPSs) | | | | | | |
| rpoB variants | 74 | −0.718 | 0.24769 | 60 | 1.540 | 0.00007* |
| rRNA methyltransferases (rRNA MTases) | 152 | 0.061 | 0.81658 | 132 | 1.049 | 0.00008* |
| beta-lactamases | 645 | 0.015 | 0.90646 | 541 | 1.004 | 2.2*10⁻¹³* |
| glycopeptide resistance clusters (RCs) | 425 | −0.721 | 0.00217 | 357 | −0.123 | 0.50527 |
| gyrA/gyrB/parC variants | 165 | −1.021 | 0.01013 | 141 | −0.979 | 0.00174 |
| non-two-component system (non- | 429 | 0.358 | 0.06540 | 388 | −0.993 | 1.3*10⁻⁷* |
| TCS) regulators | | | | | | |
| penicillin-binding proteins (PBPs) | 254 | −0.257 | 0.41414 | 211 | −1.088 | 0.00002* |
| aminoacyl-tRNA synthetases (AARSs) | 101 | −1.454 | 0.01001 | 82 | −1.094 | 0.00939 |
| efflux | 1519 | −0.215 | 0.08753 | 1346 | −1.107 | 1.7*10⁻²⁴* |
| phosphoethanolamine (pEtN) | 358 | −1.373 | 1.0*10⁻⁶* | 316 | −1.485 | 2.1*10⁻¹¹* |
| transferases | | | | | | |
| other | 410 | −0.218 | 0.34748 | 362 | −1.811 | 7.4*10⁻¹⁷* |
| two-component system (TCS) regulators | 494 | −0.900 | 0.00005* | 429 | −1.988 | 1.6*10⁻²²* |
| cya variants | 57 | −0.544 | 0.45547 | 50 | −3.273 | 0.00002* |
| porins | 74 | −1.595 | 0.01988 | 69 | −3.762 | 4.8*10⁻⁸* |

The 925 multispecies AMR genes and 830 AMR alleles were predominantly shared within species of the same phylogenetic class, especially within Gammaproteobacteria (FIG. 3C). Only 68 (7.4%) multispecies AMR genes and 38 (4.6%) alleles spanned more than one class. Of these, just 8 genes and 5 alleles were observed in at least 10 genomes in each of at least two different phylogenetic classes (FIG. 3D). These 8 multi-class genes are functionally varied, including TEM family beta-lactamases (blaTEMs), ribosomal protection proteins tetM, tetO, and tet(W/N/W), 23S rRNA methyltransferase ermB, aminoglycoside 3′-phosphotransferase aph(3′)-IIIa, and lincosamide nucleotidyltransferase lnuG. The blaTEMs were observed exclusively on plasmids, while all other multi-class AMR genes were observed on both plasmid and chromosomal DNA. Given the prevalence of blaTEMs, a case study was conducted on the distribution of complete blaTEM alleles across all species. One variant, TEM-116, was found in gram-positive strains (11 Staphylococcus aureus strains), predicted to be on a plasmid shared with Salmonella enterica strains, and found among S. aureus strains most strongly resistant to cefoxitin.

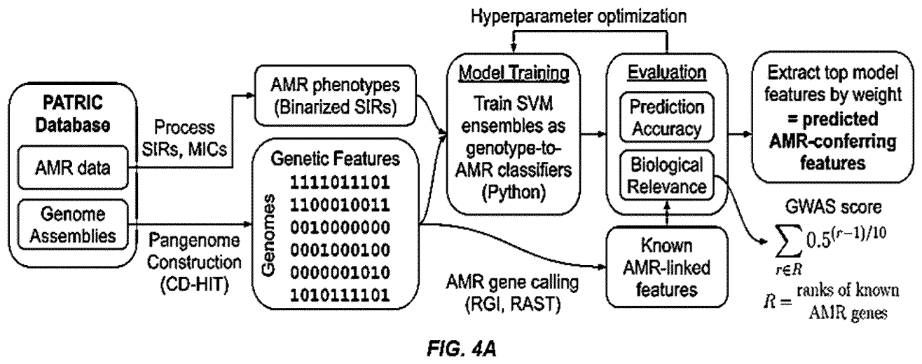

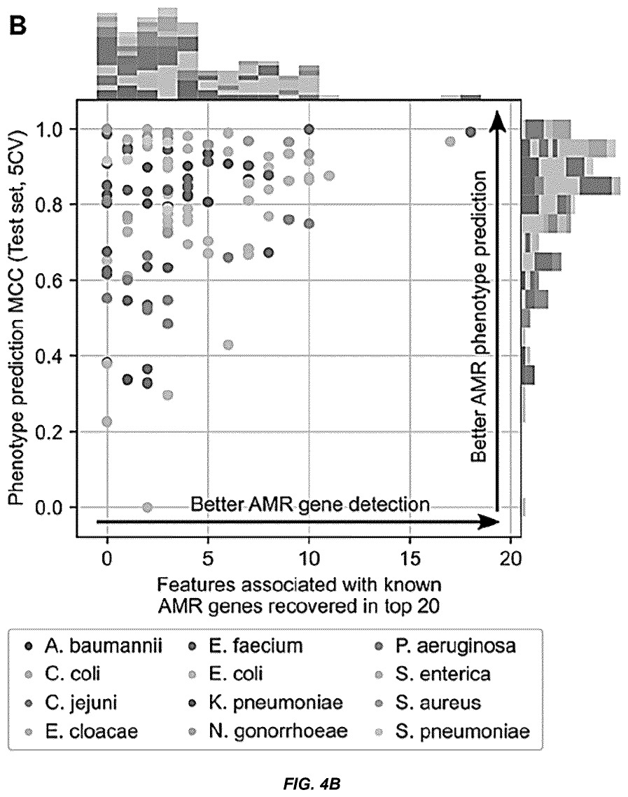

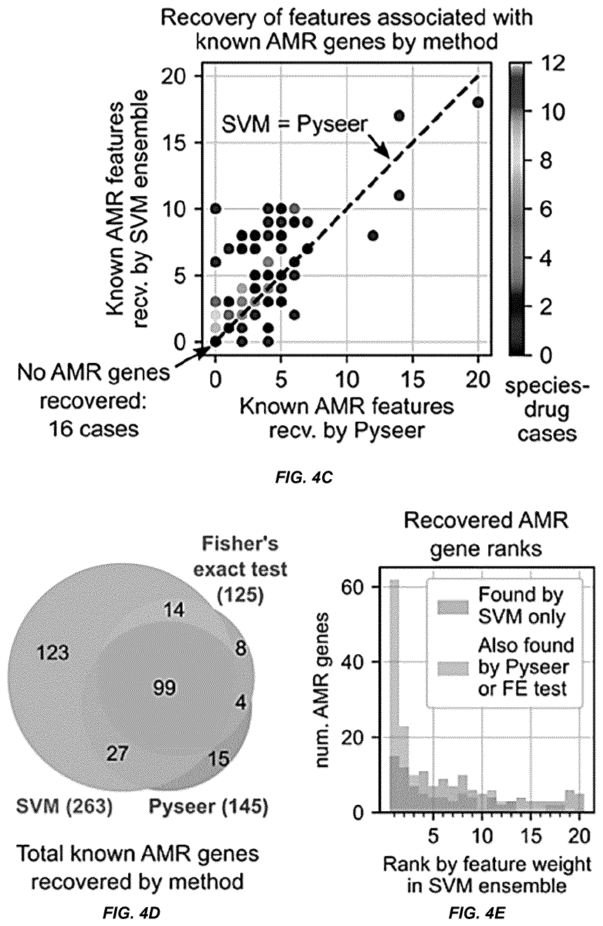

A GWAS-oriented machine learning approach for the identification of AMR-associated genes outperforms contemporary methods. To identify AMR-associated genetic features, a ML framework was developed to train models for both accuracy at predicting AMR phenotypes and biological relevance, i.e. ability to assign high feature weights to known AMR genes (FIG. 4A). For a given species-drug case, analysis was started with the support vector machine (SVM) ensemble design. SVM ensembles were trained to classify genomes as susceptible or non-susceptible based on the presence or absence of the genetic features (grouped into six types). Four hyperparameters (HPs), parameters not learned from the data but fixed in advance to control the learning process and model complexity, were varied to evaluate their impact on model performance. Ensembles under various HP combinations were evaluated over 5-fold cross validation (5CV) for 1) accuracy, as the Matthews correlation coefficient (MCC) on test set genomes to account for class imbalance, and 2) biological relevance, through a “GWAS score” defined as a weighted sum of the rankings of known AMR genetic features after sorting model features by feature weight absolute value. A feature was labeled as a known AMR genetic feature if its corresponding gene cluster was a known AMR gene for the drug of interest as annotated by RGI and PATRIC.

HP optimization was first carried out on a set of 10 species-drug test cases, testing 256 HP combinations to eliminate consistently suboptimal HP combinations before scaling to other species-drug cases (FIG. 5-6). These results were also used to assess the generalizability of the GWAS score by randomly hiding half of known AMR genes and computing correlations between GWAS scores derived from visible vs. hidden AMR genes. Results across 100 iterations of randomly hiding AMR genes suggests that the GWAS score generalizes well to hidden AMR genes, with Spearman correlation between GWAS scores from visible vs. hidden AMR genes exceeding 0.4 in 6/10 species-drug cases and 0.6 in 4/10 cases (FIG. 15).

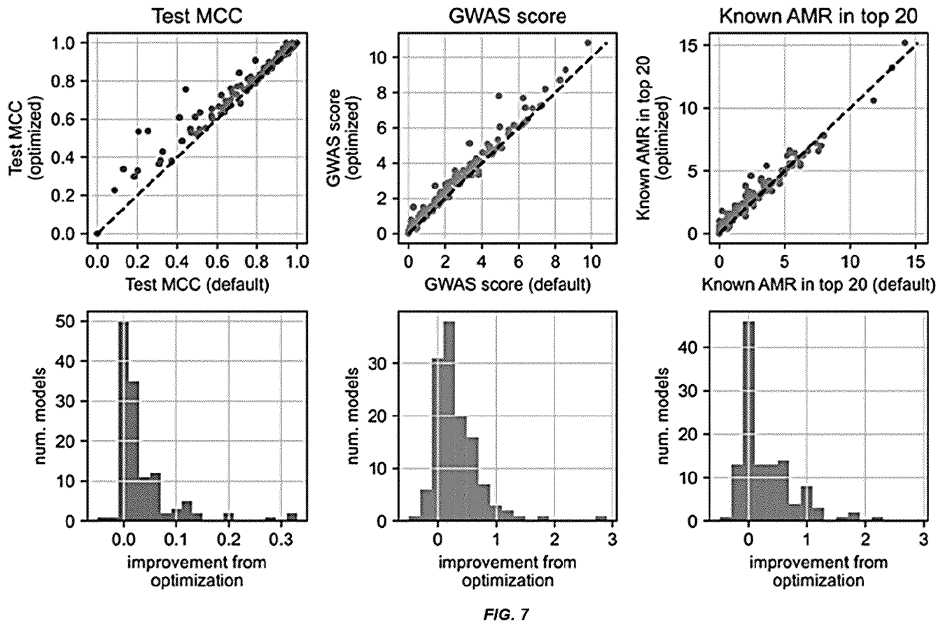

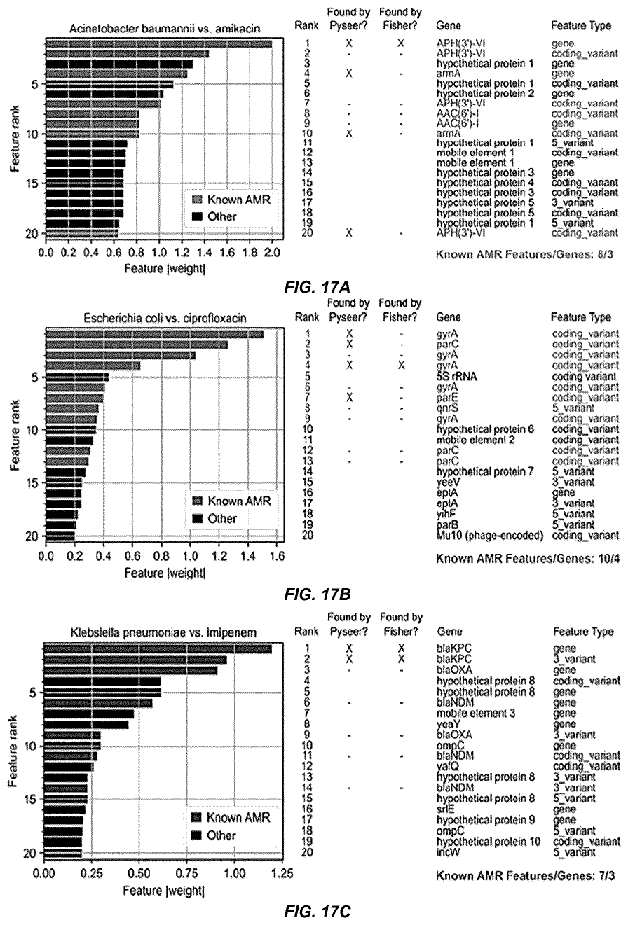

HP optimization was then applied to 127 species-drug cases with at least 100 SIRs, 10 known AMR genes, and minority phenotype >5%. For each case, models under each HP combination were ranked by mean MCC and GWAS score from 5CV, and the HP set with the highest average of the two ranks was selected as optimal. Among the final models, 41 (32%) achieved MCC >0.9 and 78 (61%) achieved MCC >0.8 on the test set during 5CV, and 103 models (81%) recovered at least one known AMR genetic feature among the top 20 features (FIG. 4B). Broadly, a high MCC was necessary but did not guarantee better recovery of known AMR genetic features, i.e. accuracy did not guarantee biological relevance. Certain dataset parameters were weakly but significantly associated with one or both performance metrics: dataset size, species, fraction of “intermediate” resistant genomes, and number of known AMR genes (n=127, Spearman R or Kruskal-Wallis test, FWER <0.05, Bonferroni correction, 12 tests) (FIG. 16, Table 9). Finally, relative to models with fixed HPs, optimizing HPs offered modest but consistent improvements to both accuracy and known AMR gene recovery. 5CV experiments showed a mean increase in test MCCs and known AMR genes recovered among the top 20 features of 0.035 and 0.230, respectively (FIG. 7). Visualizations of the top 20 model-selected features are available for three example cases (FIG. 17).

TABLE 9
Impact of input data properties on SVM ensemble performance.
Impact was assessed with Kruskal-Wallis tests for categorical
properties (species, drug class) and Spearman correlation
for quantitative properties (number of genomes, minority
phenotype fraction). Rows are sorted by p-value.

| Performance Metric | Data Metric | Statistical Test | p-value |
|---|---|---|---|
| AMR genes in top 20 | num. genomes | Spearman-R | <0.00001 |
| Test MCC in 5CV | species | Kruskal-Wallis | <0.00001 |
| AMR genes in top 20 | minority fraction | Spearman-R | 0.00464 |
| Test MCC in 5CV | num. genomes | Spearman-R | 0.00537 |
| AMR genes in top 20 | species | Kruskal-Wallis | 0.00721 |
| Test MCC in 5CV | drug class | Kruskal-Wallis | 0.30250 |
| AMR genes in top 20 | drug class | Kruskal-Wallis | 0.48732 |
| Test MCC in 5CV | minority fraction | Spearman-R | 0.95993 |

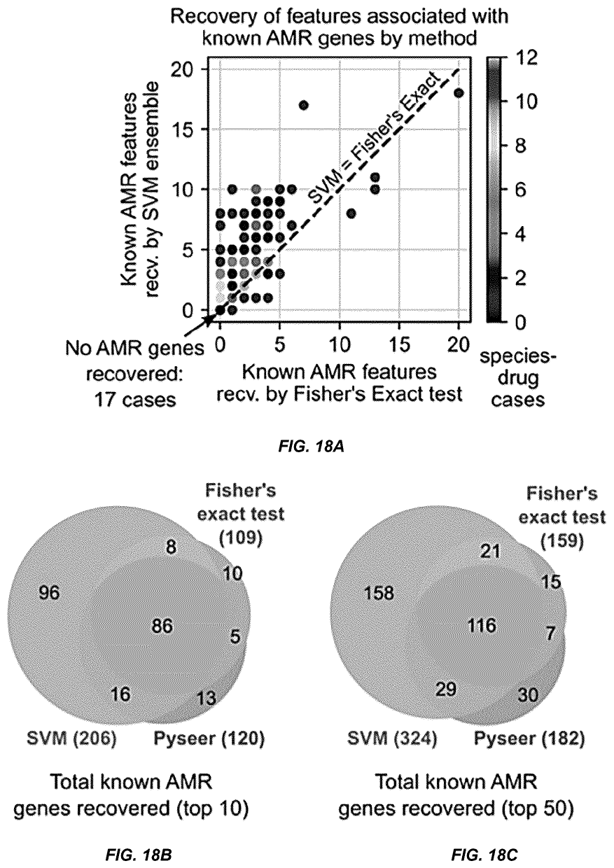

As a baseline level of AMR gene recovery, for each species-drug case, both Pyseer and Fisher's exact tests were applied to estimate the strength of association between each genetic feature and the AMR phenotype, yielding population-adjusted and unadjusted p-values, respectively. Based on the number of known AMR genetic features recovered among the top 20 features (either by feature weight for SVM or p-value otherwise), the SVM ensemble approach broadly outperformed both Pyseer and Fisher's exact test. SVM ensembles identified more known AMR-associated features than Pyseer in 73 cases (57%), the same number in 38 cases (30%), and fewer features in just 16 cases (13%) (FIG. 4C). Nearly half of the equal performance cases (16/38, 42%) were instances where neither method could recover any known AMR features, and similar differences in performance were observed when comparing SVM ensembles and Fisher's exact tests. Across all 127 cases, SVM ensembles recovered 263 known AMR gene-drug mappings of which 123 were not recovered by either Pyseer or Fisher's exact tests, compared to just 27 mappings missed by SVM ensemble but recovered by Pyseer or Fisher's exact test (FIG. 4D). Similar proportions between the number of known AMR gene-drug mappings recovered per method were observed when examining the top 10 or top 50 features (FIG. 18B-C). Examining SVM ensemble feature rankings, known AMR genes were distributed throughout the full range of ranks among the top 20 features per model, whereas those that were also recovered by other methods were concentrated among the top 3 features (95/138, 69%) with nearly half being the top weighted feature of the corresponding SVM model (62/138, 45%) (FIG. 4E). This result suggests that concordance between these three methods is mostly limited to features with the strongest statistical signals. Finally, a detailed analysis of the 30 gyrA alleles identified by SVM as associated with fluoroquinolone resistance finds all such variants to be consistent with the current literature on gyrA-mediated resistance; all positively-associated alleles carried at least one known resistance-conferring substitution, while all negatively-associated alleles carried no such mutations.

An examination of the 12 genes recovered by Fisher's exact test but not by SVM ensemble suggests several possible failure modes of the SVM approach (Table 10). First, in 4/12 cases, the missed gene is captured slightly outside of the top 20 feature threshold for recovery, with three genes ranked 21 and one ranked 33 by SVM. Second, in another 4/12 cases, many of the top features in the corresponding model were perfectly correlated, saturating the top ranks with these correlates and preventing recovery of additional AMR genes. Third, for 3/12 cases, the corresponding model was not very accurate, with mean test MCC ranging from 0.43 to 0.77. The final remaining case (arlR for ciprofloxacin resistance in S. aureus) could not be explained by these previous failure modes.

TABLE 10
Summary of known AMR gene-drug mappings recovered by Fisher's Exact test but missed by the SVM ensemble
approach. Drugs abbreviated are quinupristin-dalfopristin (Q-D) and trimethoprim-sulfamethoxazole (SXT)

| | | | Model | Rank in | Unique Features | |
|---|---|---|---|---|---|---|
| | | Missed | Test MCC | Gene | Statistically | Possible |
| Species | Drug | Gene | (5CV) | Model | in Top 50* | Failure Mode |
| Acinetobacter | tobramycin | aadA | 0.87 | 33 | 44 | Feature rank below |
| baumannii | | | | | | top 20 threshold |
| Enterobacter cloacae | cefepime | blaKPC | 0.61 | — | 46 | Poor model performance |
| Enterococcus | Q-D | eatAv | 1.00 | — | 1 | High correlation |
| faecium | | | | | | among top features |
| Enterococcus | teicoplanin | vanXA | 1.00 | 21 | 11 | Feature rank below |
| faecium | | | | | | top 20 threshold |
| Enterococcus | teicoplanin | vanA | 1.00 | 21 | 11 | Feature rank below |
| faecium | | | | | | top 20 threshold |
| Enterococcus | teicoplanin | vanHA | 1.00 | 21 | 11 | Feature rank below |
| faecium | | | | | | top 20 threshold |
| Enterococcus | vancomycin | vanZA | 0.99 | — | 16 | High correlation |
| faecium | | | | | | among top features |
| Enterococcus | vancomycin | vanYA | 0.99 | — | 16 | High correlation |
| faecium | | | | | | among top features |
| Escherichia | norfloxacin | parC | 0.43 | — | 36 | Poor model |
| coli | | | | | | performance |
| Klebsiella | SXT | sul1 | 0.84 | — | 13 | High correlation |
| pneumoniae | | | | | | among top features |
| Neisseria gonorrhoeae | erythromycin | mtrR | 0.77 | — | 48 | Poor model performance |
| Staphylococcus | ciprofloxacin | arlR | 0.97 | — | 46 | — |
| aureus | | | | | | |

*Refers to the number of features remaining among the SVM model's top 50 features by weight after collapsing perfectly correlated features together. Lower values correspond to more highly correlated features.

Identification of 142 candidate AMR-conferring genes through cross-drug and functional analysis of AMR-predictive features. Several filters were next applied to translate the best performing models to a smaller set of novel, high-confidence, AMR-conferring gene candidates (see FIG. 8A). Starting with the 78 models achieving test MCC >80%, the top 10 features from each model were filtered for those that were not already known AMR genes, occurred in at least 10 genomes with SIR data, and had both positive feature weight and LOR for resistant genomes, yielding 347 features predictive of AMR without known associations to AMR. These candidates were scored based on the number of drugs in the same class for which the feature enriches for resistance and the extent of co-occurrence with known AMR genes. Taking the top 10 features by this score for each species-drug class pair yielded 142 AMR gene candidates.

43 candidates were functionally well-characterized and consisted of four feature types: 16 genes (protein sequence clusters), 14 gene alleles (protein sequence variants), and 13 gene 5′/3′ flanking region variants (Tables 11-13). The candidates spanned 8 species and 7 drug classes and majority beta-lactam associated (26/43) due to the relative abundance of beta-lactam AMR data (FIG. 8B). The candidates span many genetic functions, and two functions occurred more than twice. Candidates related to small multidrug resistance (SMR) efflux transporters (qacE, qacEΔ1, sugE) typically associated with resistance to quaternary ammonium compounds, were linked to resistance against aminoglycosides, beta-lactams, diaminopyrimidines, and sulfonamides across four species, consistent with previous studies that find SMR transporters associated with resistance against a broad range of antibiotics beyond antiseptics33,34. Additionally, three different formate dehydrogenase genes (fdhF, fdsA, fdnG) were associated with beta-lactam resistance in Klebsiella pneumoniae, suggesting the importance of formate metabolism in AMR, possibly with respect to stress response. Finally, a majority of the sequence-variant level candidates (16/27), especially those related to flanking regions (10/13), were the most common variant of their respective gene clusters, suggesting that most observed perturbations to these genes may be deleterious with respect to AMR.

TABLE 11
16 gene clusters predicted to be associated with resistance against specific drug classes
for individual species. Accession IDs are provided for the most common sequence variant
of each gene cluster (RefSeq when possible, GenBank otherwise), along with gene names
when available and gene products. The number of resistant/susceptible genomes and log2
odds ratios (LORs) for resistance are shown for the top three drugs by LOR when data
for more than three related drugs was available. Drug class AMG refers to aminoglycoside.

| | Drug | Accession ID | Predicted | Resistant/ | |
|---|---|---|---|---|---|
| Species | Class | (Gene) | Gene Product | Susceptible | LORS |
| Acinetobacter | AMG | ENU75377.1 | Putative inner | GEN = 147/0 | GEN = 7.7 |
| baumannii | | (ydcZ) | | AMK = 144/3 | AMK = 5.8 |
| | | | membrane exporter | TOB = 114/18 | TOB = 1.9 |
| Acinetobacter | AMG | WP_000679427.1 | Small multidrug | GEN = 453/6 | GEN = 4.4 |
| baumannii | | (qacEΔ1) | resistance (SMR) | AMK = 296/170 | AMK = 1.5 |
| | | | efflux transporter | TOB = 269/163 | TOB = −0.6 |
| Acinetobacter | beta-lactam | ACC56111.1 | Uncharacterized | AMP = 82/0 | AMP = 6.5 |
| baumannii | | (yfhL) | ferredoxin-like | DOR = 13/0 | DOR = 6.1 |
| | | | protein | CFZ = 45/0 | CFZ = 5.6 |
| Enterococcus | AMG | WP_000228166.1 | Nucleotidyltransferase | STR = 89/0 | STR = 14.8 |
| faecium | | | domain containing | | |
| | | | protein | | |
| Enterococcus | glycopeptide | WP_000754864.1 | Cadmium resistance | TEC = 138/0 | TEC = 14.8 |
| faecium | | (cadC) | transcriptional | VAN = 498/150 | VAN = 3.4 |
| | | | regulatory protein | | |
| Escherichia coli | beta-lactam | WP_000243817.1 | Tryptophan synthase | CXM = 196/1 | CXM = 9.1 |
| | | | (indole-salvaging) | CTX = 236/4 | CTX = 7.7 |
| | | | | SAM = 42/0 | SAM = 7.3 |
| Escherichia coli | quinolone | WP_000598813.1 | Anaerobic C4- | CIP = 59/14 | CIP = 3.3 |
| | | (dcuC) | dicarboxylate | LVX = 31/3 | LVX = 2.1 |
| | | | transporter | | |
| Klebsiella | beta-lactam | WP_002885150.1 | Formate | CRO = 1508/11 | CRO = 5.3 |
| pneumoniae | | (fdhF) | dehydrogenase H | ETP = 130/1 | ETP = 4.0 |
| | | | | CFZ = 1487/58 | CFZ = 3.7 |
| Klebsiella | beta-lactam | AKE78078.1 | Formate | CRO = 1499/10 | CRO = 5.4 |
| pneumoniae | beta-lactam | (fdsA) | dehydrogenase H | CFZ = 1486/40 | CFZ = 4.4 |
| | | | | ETP = 130/1 | ETP = 4.0 |
| Klebsiella | | EWF54733.1 | Formate | CRO = 1494/10 | CRO = 5.4 |
| pneumoniae | | (fdnG) | dehydrogenase N | ETP = 128/1 | ETP = 4.0 |
| | | | alpha subunit | CFZ = 1478/49 | CFZ = 3.9 |
| Klebsiella | beta-lactam | AHG50656.1 | 3-oxo-tetronate | CRO = 71/0 | CRO = 6.3 |
| pneumoniae | | (ygbK) | kinase | AMP = 62/0 | AMP = 6.0 |
| | | | | CAZ = 107/1 | CAZ = 4.0 |
| Klebsiella | diamino- | WP_000679427.1 | Small multidrug | TMP = 10/0 | TMP = 6.2 |
| pneumoniae | pyrimidine | (qacEA1) | resistance (SMR) | SXT = 855/42 | SXT = 3.6 |
| | | | efflux transporter | | |
| Salmonella | sulfonamide | WP_000800531.1 | Small multidrug | SXT = 11/4 | SXT = 6.6 |
| enterica | | (qacE) | resistance (SMR) | SIX = 15/0 | SIX = 5.4 |
| | | | efflux transporter | SMZ = 12/4 | SMZ = 0.4 |
| Staphylococcus | beta-lactam | WP_000872606.1 | MaoC domain protein | MET = 202/4 | MET = 16.9 |
| aureus | | (maoC) | | FOX = 786/0 | FOX = 16.2 |
| | | | | OXA = 29/3 | OXA = 15.3 |
| Staphylococcus | beta-lactam | WP_000616816.1 | Cadmium resistance | PEN = 461/28 | PEN = 2.0 |
| aureus | | (cadD) | transporter | BPG = 82/4 | BPG = 0.1 |
| | | | | FOX = 330/165 | FOX = −1.5 |
| | | | | FOX = 661/0 | FOX = 12.3 |
| Staphylococcus | beta-lactam | WP_001186608.1 | Site-specific | MET = 198/58 | MET = 8.9 |
| aureus | | | recombinase | OXA = 8/13 | OXA = 3.3 |

TABLE 12
14 gene coding variants predicted to be associated with resistance against specific drug classes for individual
species. Accession IDs are provided for the exact sequence variant (RefSeq when possible, GenBank otherwise),
along with gene names when available, gene products, and mutations with respect to the most observed variant.
The number of resistant/susceptible genomes and log₂ odds ratios (LORs) for resistance are shown for the
top three drugs by LOR when data for more than three related drugs was available.

| | Drug | Accession ID | Predicted | | Resistant/ | |
|---|---|---|---|---|---|---|
| Species | Class | (Gene) | Gene Product | Muts.* | Susceptible | LORs |
| Acinetobacter | quinolone | WP_000586912.1 | Lipopolysaccharide | — | CIP = 795/166 | CIP = 3.0 |
| baumannii | | (lptF) | export system | | LVX = 731/143 | LVX = 2.5 |
| | | | permease protein | | | |
| Acinetobacter | tetracycline | ADX03353.1 | Biotin carboxylase | — | TET = 107/0 | TET = 7.4 |
| baumannii | | (bccA) | | | MIN = 86/20 | MIN = 4.0 |
| Campylobacter | quinolone | AHK72934.1 | Ribonuclease Y | d1-86, | NAL = 41/16 | NAL = 2.9 |
| coli | | (rny)** | | V87M | CIP = 39/18 | CIP = 2.6 |
| Escherichia coli | beta-lactam | WP_000811566.1 | Putative | — | FOX = 46/0 | FOX = 9.4 |
| | | (yjfN) | uncharacterized | | CTT = 4/15 | CTT = 4.5 |
| | | | protein, DUF1471 | | CXM = 246/136 | CXM = 3.2 |
| Escherichia coli | beta-lactam | WP_000118520.1 | Small multidrug | T37A, M85A, | FOX = 40/0 | FOX = 9.0 |
| | | (sugE) | resistance (SMR) | A88L, A91G, | CAZ = 101/1 | CAZ = 7.7 |
| | | | efflux transporter | L95A, +13 | CRO = 85/0 | CRO = 7.5 |
| Escherichia coli | beta-lactam | WP_001588947.1 | Fumarate | V111D | AMC = 19/1 | AMC = 4.7 |
| | | (frdD)*** | reductase | | AMP = 14/0 | AMP = 4.5 |
| | | | subunit D | | CXM = 12/1 | CXM = 4.4 |
| Escherichia coli | quinolone | WP_000228346.1 | D-serine, D- | — | LVX = 251/35 | LVX = 4.0 |
| | | (cycA)*** | alanine, glycine | | CIP = 573/468 | CIP = 3.0 |
| | | | transporter | | NAL = 41/17 | NAL = 0.7 |
| Klebsiella | beta-lactam | WP_004183775.1 | Cold shock protein | V54A, H55L, | TIM = 16/2 | TIM = 7.0 |
| pneumoniae | | | of CSP family | A57T, Q62P, +2 | AMP = 107/0 | AMP = 6.8 |
| | | | | | CEF = 13/0 | CEF = 4.1 |
| Salmonella | beta-lactam | WP_001221666.1 | Lipocalin Blc | L49F, S98D, | CRO = 302/0 | CRO = 13.6 |
| enterica | | (blc) | | S175P, +10 | AMC = 301/0 | AMC = 12.1 |
| | | | | | CTF = 297/3 | CTF = 10.9 |
| Salmonella | beta-lactam | WP_000118520.1 | Small multidrug | T37A, | CRO = 304/0 | CRO = 13.7 |
| enterica | | (sugE) | resistance (SMR) | A104T, +9 | AMC = 303/0 | AMC = 12.1 |
| | | | efflux transporter | | CTF = 299/3 | CTF = 11.0 |
| Staphylococcus | beta-lactam | WP_000872606.1 | MaoC domain | — | OXA = 29/3 | OXA = 15.3 |
| aureus | | (maoC) | protein | | MET = 201/4 | MET = 14.5 |
| | | | | | FOX = 713/0 | FOX = 13.1 |
| Staphylococcus | beta-lactam | WP_000958858.1 | Glycerophosphoryl- | — | OXA = 29/3 | OXA = 15.3 |
| aureus | | | diester | | FOX = 775/0 | FOX = 15.2 |
| | | | phosphodiesterase | | MET = 201/3 | MET = 14.8 |
| Streptococcus | beta-lactam | WP_000248982.1 | Cell wall surface | V66I, D111E, | AMX = 13/2 | AMX = 13.1 |
| pneumoniae | | | anchor family | S160G, E172K | MEM = 15/0 | MEM = 10.1 |
| | | | protein | | CXM = 15/0 | CXM = 9.7 |
| Streptococcus | beta-lactam | WP_000203066.1 | Glutathione | A80T, S132G | AMX = 13/3 | AMX = 12.7 |
| pneumoniae | | (gpo) | peroxidase | | MEM = 16/0 | MEM = 10.6 |
| | | | | | CXM = 16/0 | CXM = 10.3 |

*When more than four mutations are detected, only mutations with BLOSUM62 score ≤ 0 are shown and the number of additional mutations is listed at the end. If no mutations are shown, the variant of interest is the most commonly observed variant.
**No exact variant was found on GenBank. AHK72934.1 refers to the most common variant of the gene, while the variant of interest contains the 86-amino acid N-terminal truncation “d1-86”.
***Selected for experimental validation.

TABLE 13
13 gene flanking noncoding variants predicted to be associated with resistance against specific drug classes for individual
species. Accession IDs are provided for the most common coding variant of the corresponding gene cluster (RefSeq when
possible, GenBank otherwise), along with gene names when available and gene products. Mutations are defined relative to
the most common 5′/3′ variant for the corresponding gene. The number of resistant/susceptible genomes and log₂
odds ratios (LORs) for resistance are shown for the top three drugs by LOR when data for more than three related
drugs was available.

| | | | | | | |
|---|---|---|---|---|---|---|
| | Drug | | Predicted | | Resistant/ | |
| Species | Class | Accession (Gene) | Gene Product | Mutations* | Susceptible | LORs |

5′ flanking variants

| | | | | | | |
|---|---|---|---|---|---|---|
| Acinetobacter | beta-lactam | AGQ10471.1 | Coenzyme PQQ | −12_2delTGATTTA | CTX = 73/0 | CTX = 6.4 |
| baumannii | | (pqqA) | synthesis | ATCAAGTG** | CRO = 73/0 | CRO = 6.4 |
| | | | protein A | | AMP = 73/0 | AMP = 6.4 |
| Acinetobacter | tetracycline | WP_000096554.1 | T6SS AAA+ | Most common | TET = 241/12 | TET = 3.3 |
| baumannii | | (clpV) | chaperone | variant | MIN = 71/18 | MIN = 2.3 |
| Campylobacter | quinolone | WP_002805020.1 | GNAT acetyl- | Most common | CIP = 94/71 | CIP = 6.0 |
| coli | | | transferase | variant | NAL = 95/70 | NAL = 5.5 |
| Campylobacter | quinolone | WP_002783313.1 | Putative | Most common | NAL = 97/115 | NAL = 5.5 |
| coli | | | transmembrane | variant | CIP = 95/117 | CIP = 5.4 |
| | | | transport | | | |
| | | | protein, MFS | | | |
| Escherichia | beta-lactam | AAG56074.1 | Small inner | −19A > C, −23A > | CTX = 28/0 | CTX = 6.7 |
| coli | | (ymgF) | membrane | G, −25_24delTC, | CAZ = 30/1 | CAZ = 5.7 |
| | | | protein | −30insA, −48G > | CXM = 14/1 | CXM = 4.6 |
| | | | | A, −173A > T, −175C > | | |
| | | | | A, −211A > T, −213C > T | | |
| Escherichia | quinolone | WP_000017703.1 | Ni/Fe- | Most common | LVX = 260/56 | LVX = 3.3 |
| coli | | (hybB) | hydrogenase 2 | variant | CIP = 606/606 | CIP = 2.8 |
| | | | b-type | | NAL = 45/16 | NAL = 1.4 |
| | | | cytochrome | | | |
| | | | subunit | | | |
| Streptococcus | beta-lactam | WP_000449822.1 | Thiaminase II | Most common | AMX = 13/4 | AMX = 12.4 |
| pneumoniae | | | | variant | MEM = 16/1 | MEM = 9.6 |
| | | | | | CXM = 16/1 | CXM = 9.3 |
| Streptococcus | beta-lactam | ADI69655.1 | Neopullulanase | Most common | AMX = 13/3 | AMX = 12.7 |
| pneumoniae | | (nplT) | | variant | MEM = 15/1 | MEM = 9.1 |
| | | | | | CXM = 15/1 | CXM = 8.6 |
| Streptococcus | beta-lactam | WP_000592948.1 | Unsaturated | −186C > T | AMX = 13/6 | AMX = 11.9 |
| pneumoniae | | (ugl) | chondroitin | | CXM = 17/2 | CXM = 9.6 |
| | | | disaccharide | | MEM = 17/3 | MEM = 9.2 |
| | | | hydrolase | | | |

3′ flanking variants

| | | | | | | |
|---|---|---|---|---|---|---|
| Campylobacter | quinolone | WP_002777456.1 | Transcriptional | Most common | NAL = 98/115 | NAL = 7.4 |
| coli | | (hspR) | repressor of | variant | CIP = 96/117 | CIP = 7.4 |
| | | | DnaK operon | | | |
| Klebsiella | beta-lactam | WP_000679427.1 | Small multidrug | Most common | AMP = 759/0 | AMP = 10.3 |
| pneumoniae | | (qacEA1) | resistance | variant | CEF = 27/0 | CEF = 5.5 |
| | | | (SMR) efflux | | CRO = 772/4 | CRO = 4.3 |
| | | | transporter | | | |
| Salmonella | quinolone | WP_012772747.1 | Psp operon | Most common | NAL = 9/11 | NAL = 3.5 |
| enterica | | | transcriptional | variant | CIP = 9/10 | CIP = 3.2 |
| | | | activator | | | |
| Streptococcus | beta-lactam | WP_000145597.1 | Neopullulanase | Most common | AMX = 13/3 | AMX = 12.7 |
| pneumoniae | | (nplT) | | variant | MEM = 15/1 | MEM = 9.1 |
| | | | | | CXM = 15/1 | CXM = 8.6 |

*Mutations are denoted relative to the start codon for 5′ variants. Position −1 corresponds to the first base pair immediately adjacent on the 5′ side of the gene's start codon.
**Results in the deletion of a GTG start codon and the 12 base pairs immediately adjacent on the 5′ side, with respect to the most common variant. The candidate variant begins with an ATG start codon.

Two allele candidates related to E. coli core genes were selected for experimental validation: The wildtype D-serine/D-alanine/glycine transporter (cycA) allele associated with quinolone resistance, and fumarate reductase subunit D (frdD) allele with a V111D substitution associated with beta-lactam resistance. E. coli BW25113 was chosen as the base strain for validation to make use of the Keio knockout collection36, which is genetically identical to K12 MG1655 across all positions within 40 kb of frdD and cycA based on reference genomes NZ_CP009273.1 and U00096.3. Wildtype (WT) cycA and frdD were defined as the most common allele of the respective genes observed across all E. coli genomes in this study, which were also the alleles present in the BW25113 and K-12 MG1655 reference genomes.

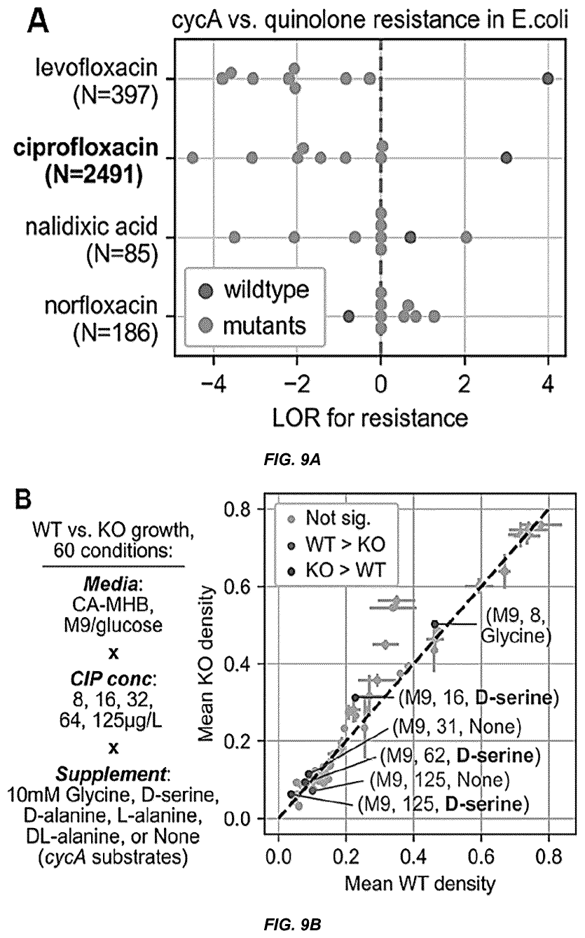

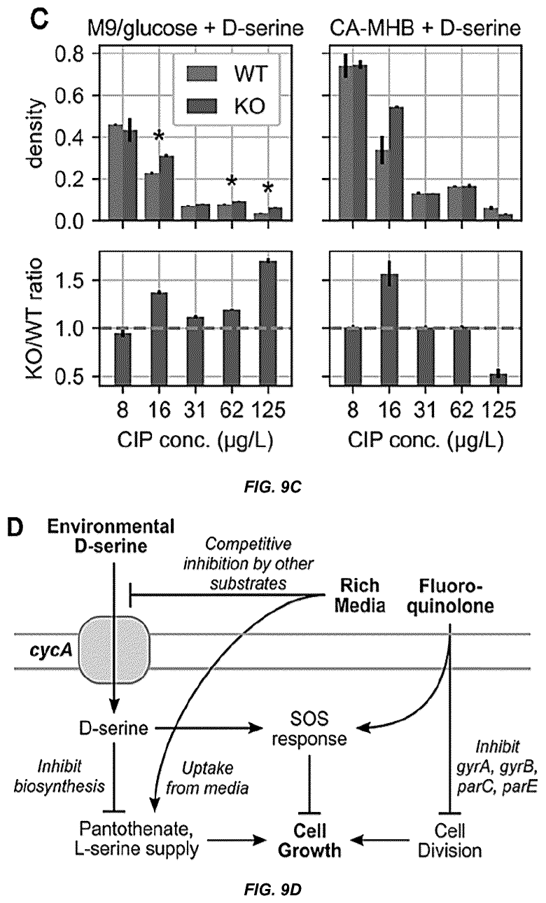

Experimental validation 1: Loss of amino acid transporter CycA confers limited quinolone resistance in minimal media with D-serine. The WT cycA variant, the fifth highest weighted feature in the E. coli AMR model for levofloxacin, had the highest LOR for resistance among all cycA variants in 2/4 quinolone drugs (see FIG. 9A). To assess the impact of cycA on quinolone resistance, maximum cell density was measured for BW25113 (WT) and corresponding ΔcycA mutant (KO, from the Keio collection) under 60 conditions based on three variables: concentration of ciprofloxacin (CIP), supplementation with known substrates of the D-serine/D-alanine/glycine transporter encoded by cycA, and choice of rich vs. minimal media (cation-adjusted Mueller-Hinton Broth CA-MHB vs. M9 media with glucose). 6/60 tested conditions resulted in significantly different final densities between WT and KO (Welch t-test, FDR <0.05, Benjamini-Hochberg correction), three of which involved D-serine and M9 media (see FIG. 9B). Across all conditions involving D-serine, while increasing CIP concentration reduced final density for both strains, the KO strain achieved higher densities for 16-125 μg/L CIP, but only in M9 media and not in CA-MHB (see FIG. 9C).

One explanation consistent with this conditional increase in CIP resistance by cycA KO involves the toxicity of D-serine, it's transported by CycA, and interaction with quinolones through the SOS response (see FIG. 9D). D-serine, which inhibits L-serine and pantothenate biosynthesis, can be bacteriostatic in minimal media. D-serine uptake can be impaired by cycA KO but also through competitive inhibition of CycA by other substrates in rich media, and its toxicity mitigated by direct uptake of L-serine and pantothenate in rich media. With respect to CIP, both D-serine and fluoroquinolones induce SOS response but to differing extents. As systematic alterations in SOS response induction have been shown to reduce fluoroquinolone resistance, the presence of both D-serine and CIP may result in a SOS response that is adapted to neither stress and consequently results in greater susceptibility to CIP.

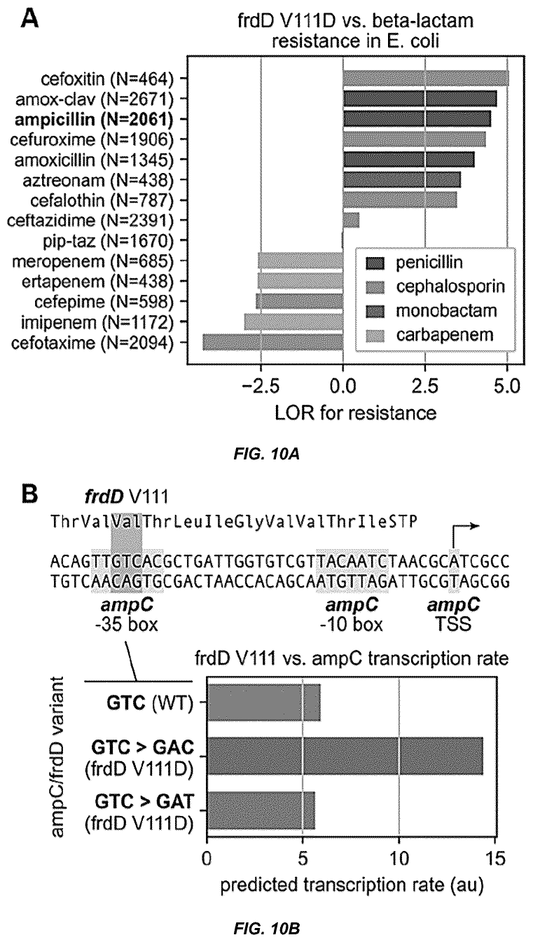

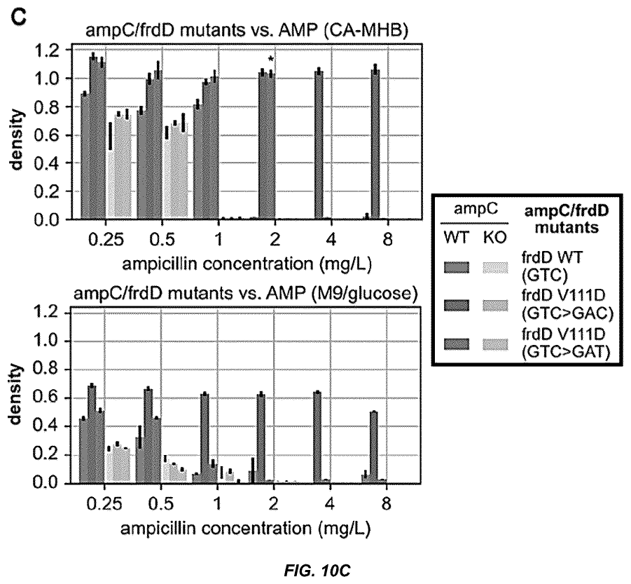

Experimental validation 2: The V111D substitution in frdD confers beta-lactam resistance solely through altering expression of the overlapping beta-lactamase gene ampC. Next, the frdD V111D variant was selected for validation as it was the third highest weighted feature in the E. coli AMR model for ampicillin, in the top 10 for four E. coli beta-lactam models and enriched for resistant strains (LOR >3) in 7/14 beta-lactam drugs with AMR data (see FIG. 10A). Given the proximity of frdD to the adjacent beta-lactamase gene ampC, this mutation occurs in the −35 box of the ampC promoter and coincides with ampC overexpression mutations known to increase beta-lactam resistance (see FIG. 10B). Appropriately, an ampC 5′ variant containing the equivalent mutation was ranked 5th in the model for amoxicillin-clavulanate, though no other 5′ variants with the mutation were in the top 50 hits of any other beta-lactam model. To assess whether frdD V111D is simply a byproduct of an ampC promoter mutation or contributes separately to beta-lactam resistance, two frdD mutations were examined both resulting in the V111D substitution but with different effects on ampC transcription as predicted by Promoter Calculator: (1) 332T>A, predicted to increase ampC transcription 2.6-fold, or (2) 332TC>AT, predicted to have minimal effect on ampC (see FIG. 10B). Six E. coli strains were examined, based on frdD variants (WT or either mutation) generated in either BW25113 or corresponding ΔampC mutant (from the Keio collection). Maximum cell density achieved by these strains were measured under increasing concentrations of ampicillin in either rich (CA-MHB) or minimal (M9+glucose) media.

Across all strains, only the mutant with both ampC and the overexpression mutation was able to grow at ≥2 mg/L ampicillin in either media, and final densities were not impacted even at 8 mg/L (see FIG. 10C). The mutant with ampC and the non-overexpressing frdD mutation grew at 2 mg/L in CA-MHB only, but this growth was delayed by at least 8 hours in all three replicates, suggesting some degree of susceptibility. Furthermore, all AampC mutants failed to grow at 2 mg/L ampicillin regardless of frdD status and reached lower densities than their corresponding ampC WT strain in 17/18 conditions with <2 mg/L ampicillin and significantly so in 15/18 cases (FDR <0.05, Welch t-test, Benjamini-Hochberg correction); the only exception was between strains with WT frdD at 1 mg/L ampicillin in M9 in which both the ampC and ampC KO strains exhibited very little growth (OD<0.1). These results suggest that the frdD V111D substitution requires ampC to confer beta-lactam resistance and is unlikely to contribute to resistance through any ampC-independent mechanism.

A number of embodiments have been described herein. Nevertheless, it will be understood that various modifications may be made without departing from the spirit and scope of this disclosure. Accordingly, other embodiments are within the scope of the following claims.

1. A computer-implemented method to identify novel genetic features that are predictive of a phenotype based upon a genome collection indicative of a genus, family or species of organism of interest from a dataset of genome assemblies and corresponding measurements related to the phenotype, comprising carrying out steps (1-4) and optionally, step 1′:
(1) enumerating genetic variation through pangenome construction from genome assemblies;
(1′) identifying genetic features known to be associated with a phenotype to supplement the machine learning models of steps (2)-(3);
(2) training machine learning models to predict phenotypes from the genetic variation enumerated in step (1) and evaluating the machine learning model for best performing hyperparameter (HP) ranges predictive of a phenotype from a genetic feature;
(3) re-training the machine learning model of step (2) with best performing hyperparameter (HP) ranges and sorting genetic features that are most predictive of a phenotype based upon a genome collection indicative of a genus, family or species of organism of interest in order to generate a defined, ranked set of genetic features predictive of a phenotype based upon a genome collection indicative of a genus, family or species of organism of interest; and
(4) identifying novel genetic features that are predictive of a phenotype based upon a genome collection indicative of a genus, family or species of organism of interest by removing features already known to be predictive of a phenotype based upon a genome collection indicative of a genus, family or species of organism of interest from the defined, ranked set of genetic features in step (3).

2. The computer-implemented method of claim 1, wherein the phenotype of interest is antimicrobial resistance.

3. The computer-implemented method of claim 1, wherein the dataset comprises genome assemblies from microbes.

4. The computer-implemented method of claim 1, wherein for step (1), the genome assemblies are first processed by carrying out steps (a)-(d), and optionally, step (d′):
(a) identifying genetic features in each genome assembly;
(b) dividing identified genetic features into protein coding sequences (CDSs) and noncoding features;
(c) computing the number of CDSs, number of contigs, and total length of each genome assembly in the dataset;
(d) identifying genomes assemblies that meet defined genetic feature criteria by filtering out genome assemblies from the dataset that do not satisfy one or more of the following criteria:
(i) the number of contigs is within 2.5 times the median across all genome assemblies,
(ii) the number of CDs is within 3 standard deviations of the mean across all genome assemblies, and
(iii) the total genome length is within 3 standard deviations of the mean across all genome assemblies; and
(d′) filtering the identified genome assemblies of (d) to filter out genomes that have an EvalCon fine consistency less than 90%, less than 88%, less than 87%, less than 85% or less than 80%.

5. The computer-implemented method of claim 4, wherein genome assemblies are filtered out from the dataset if they do not meet criteria (i), (ii) and (iii).

6. The computer-implemented method of claim 1, wherein for step (1), the pangenomes are constructed and genetic variation is enumerated thereof, by carrying out steps (A)-(F) and optionally step (D′):
(A) reducing the set of all identified CDSs across all genome assemblies to a non-redundant set of unique CDSs;
(B) clustering all unique CDSs by amino acid sequence;
(C) enumerating every CDS cluster, and every sequence variant of each CDS cluster, wherein the enumerated CDS clusters are referred to as “genes”, and wherein sequence variants of each enumerated CDS cluster are referred to as “gene coding variants” of the corresponding gene;
(D) identifying unique noncoding sequences flanking occurrences of each gene;
(D′) if noncoding nucleic acid sequence features are available, then steps (A)-(C) are repeated for noncoding nucleic acid sequences to yield enumerated noncoding sequence clusters and variants, wherein the enumerated noncoding clusters are referred to as “noncoding features”, and wherein sequence variants of each enumerated noncoding cluster are referred to as “noncoding variants;”
(E) determining the presence/absence of features (1)-(4) and optionally (5) and (6) for all genome assemblies: (1) genes, (2) gene coding variants, (3) gene 5′ variants, (4) gene 3′ variants, (5) noncoding features, and (6) noncoding variants; and
(F) encoding the genetic variation of the dataset as a (genome assembly x feature) binary matrix of these presence/absence calls.

7. The computer-implemented method of claim 1, wherein for step (D), the unique noncoding sequences flanking the occurrences of the gene are identified by:
(i) identifying the locations of gene coding variants across genome assemblies;
(ii) extracting 300 bp sequences immediately upstream and/or downstream at each location;
(iii) removing sequences shorter than 300 bp due to contig boundaries; and
(iv) reducing all upstream and/or downstream sequences into non-redundant sets of 300 bp nucleic acid sequences, wherein the non-redundant sets of 300 bp nucleic acid sequences are referred to as gene 5′ variants and gene 3′ variants, respectively.

8. The computer-implemented method of claim 1, wherein the computer-implemented method includes step (1′), and wherein the genetic features known to be associated with a phenotype to supplement the machine learning models of step (2) are identified by carrying out steps (I)-(IV):
(I) Identifying the most common coding variant for each gene, and annotating the variants for phenotype-associated genes;
(II) Extracting the phenotype-associated genes based upon defined criteria;
(III) Identifying other genes that have same defined criteria as the phenotype-associated genes, and annotate these other genes as phenotype-associated genes; and
(IV) Labelling all phenotype-associated genes, as well as their associated coding variants, 5′ variants, and 3′ variants as genetic features that are known to be associated with a phenotype and species of interest.

9. The computer-implemented method of claim 8, wherein the phenotype-associated genes contribute to antimicrobial resistance.

10. The computer-implemented method of claim 9, wherein the variants are annotated as antimicrobial resistance (AMR) genes using RGI from CARD.

11. The computer-implemented method of claim 10, wherein the genes annotated as AMR genes are assigned an ARO ID, and are extracted based upon the criteria of being associated with a drug of interest based upon the CARD ontology, and wherein the extraction process comprises:
(aa) initializing a graph with each ARO ID from the CARD ontology corresponding to a node, and adding an edges U→V whenever:
(i) U is a gene and has relationship “is_a” to V;
(ii) V is a drug and has relationship “is_a” to U;
(iii) V has relationship “has_part” to U;
(iv) U has any of the following relationships to V: “part_of”,
“regulates”, “confers_resistance_to_antibiotic”,
“confers_resistance_to_drug_class”; and
(bb) filtering the initial identified AMR genes to whose ARO ID has a direct path to the node corresponding to the drug of interest in the graph.

12. The computer-implemented method of claim 1, wherein for step (2), the machine learning model comprises the following methodology:
identifying the top 50,000 genetic features by individual association with the phenotype of interest, sorting by log odds ratio (LOR), and acquiring the top 25,000 and bottom 25,000 genetic features;
defining the machine learning model and hyperparameter (HP) ranges to use in predicting the phenotype;
training and evaluating the model and HP combinations over x-fold cross validation, wherein the accuracy for an HP combination is defined as the mean Matthews correlation coefficient (MCC) on the test set across the x-folds; and
selecting the HP combination that yields the best performance over the x-fold cross validation, wherein the HP combination with the highest mean MCC is selected.

13. The computer-implemented method of claim 12, wherein the machine learning model uses 5-fold cross validation.

14. The computer-implemented method of claim 12, wherein the machine learning model is a support vector machine (SVM) ensembles implemented in scikit-learn.

15. The computer-implemented method of claim 12, wherein the HP ranges are defined as follows:
(aaa) number of estimators=25, 50, 100, 200;
(ii) fraction of samples per estimator=25%, 50%, 75%, 100%;
(bbb) fraction of features per estimator=25%, 50%, 75%, 100%; and
(ccc) C (SVM regularization term)=0.1, 1, 10, 100.

16. The computer-implemented method of claim 12, wherein if the computer-implemented model includes step (1′), then evaluating how the known genetic features were recovered by the machine learning model at each fold using the following:
(xx) for a trained model at each fold, computing each genetic feature's weight as the absolute value of the average of coefficients assigned to a genetic feature across all individual SVMs in the ensemble that had access to the feature, when accounting from genetic feature subsampling;
(yy) sorting and ranking all features by weight, and computing a model “GWAS Score” from the ranks “r” of known phenotype-associated features using EQ. 1:
$$GWASScore = {\sum }_{r ∈ known} 0 . {5}^{( r - 1 ) / 10} \qquad ( Eq . 1 )$$
wherein rank=1 corresponds to the highest weight; and
(zz) computing the mean GWAS Score across the x folds as the GWAS Score of the HP combinations.

17. The computer-implemented method of claim 16, wherein if known features were annotated, then each HP combination is ranked by mean MCC and separately by mean GWAS Score and selecting the HP combination with the highest sum of ranks, wherein rank=1 corresponds to the highest MCC or GWAS Score.

18. The computer-implemented method of claim 1, wherein the machine learning model predicts a binary phenotype variable.

19. The computer-implemented method of claim 1, wherein the machine learning model is modified to predict categorical or continuous/numerical variables.
